SEQUENCE LISTING (24 sequences)

SEQ ID NO: 1 - DNA, 20 bp, ARTIFICIAL SEQUENCE
  Primer
cagcatgtgtctgccactct

SEQ ID NO: 2 - DNA, 20 bp, ARTIFICIAL SEQUENCE
  Primer
gtgttgtcccaagatgcctt

SEQ ID NO: 3 - DNA, 20 bp, ARTIFICIAL SEQUENCE
  Primer
cagaccaacatctcccacct

SEQ ID NO: 4 - DNA, 20 bp, ARTIFICIAL SEQUENCE
  Primer
ggttcccaactctagcacca

SEQ ID NO: 5 - DNA, 20 bp, ARTIFICIAL SEQUENCE
  Primer
gttctctgggaaatcgtgga

SEQ ID NO: 6 - DNA, 20 bp, ARTIFICIAL SEQUENCE
  Primer
tggtcttggtccttagccac

SEQ ID NO: 7 - DNA, 20 bp, ARTIFICIAL SEQUENCE
  Primer
tccgaagaggccagaaacta

SEQ ID NO: 8 - DNA, 20 bp, ARTIFICIAL SEQUENCE
  Primer
cacgggtagcaagaacaggt

SEQ ID NO: 9 - DNA, 20 bp, ARTIFICIAL SEQUENCE
  Primer
agcacagcagatgtgaatgc

SEQ ID NO: 10 - DNA, 20 bp, ARTIFICIAL SEQUENCE
  Primer
cctcttccttcatgtcaggc

SEQ ID NO: 11 - DNA, 20 bp, ARTIFICIAL SEQUENCE
  Primer
tttcactctcggtccacctc

SEQ ID NO: 12 - DNA, 20 bp, ARTIFICIAL SEQUENCE
  Primer
agatgcttgacgttggctct

SEQ ID NO: 13 - DNA, 21 bp, ARTIFICIAL SEQUENCE
  Primer
ctacactcttgaccctgacac

SEQ ID NO: 14 - DNA, 21 bp, ARTIFICIAL SEQUENCE
  Primer
gccacgataacaatcttgtcc

SEQ ID NO: 15 - DNA, 20 bp, ARTIFICIAL SEQUENCE
  Primer
tcaaatcccaccagtgttga

SEQ ID NO: 16 - DNA, 20 bp, ARTIFICIAL SEQUENCE
  Primer
gggtccaattaatcccgagt

SEQ ID NO: 17 - DNA, 20 bp, ARTIFICIAL SEQUENCE
  Primer
agccatgtacgtagccatcc

SEQ ID NO: 18 - DNA, 20 bp, ARTIFICIAL SEQUENCE
  Primer
ctctcagctgtggtggtgaa

SEQ ID NO: 19 - DNA, 20 bp, ARTIFICIAL SEQUENCE
  Primer
tcaaatcccaccagtgttga

SEQ ID NO: 20 - DNA, 20 bp, ARTIFICIAL SEQUENCE
  Primer
gggtccaattaatcccgagt

SEQ ID NO: 21 - DNA, 22 bp, ARTIFICIAL SEQUENCE
  Primer
gcctgggcttatttctctgatg

SEQ ID NO: 22 - DNA, 22 bp, ARTIFICIAL SEQUENCE
  Primer
tgagcaggattcagtgtgttcg

SEQ ID NO: 23 - DNA, 20 bp, ARTIFICIAL SEQUENCE
  Primer
cctcgaccacaactgcttag

SEQ ID NO: 24 - DNA, 18 bp, ARTIFICIAL SEQUENCE
  Primer
gggtggcagtgatggcat

(19) United States
(12) Patent Application Publication
(10) Pub. No.: US 2011/0150843 A1
(43) Pub. Date: Jun. 23, 2011

(54) METHOD FOR THE THERAPEUTIC CORRECTION OF HEMOPHILIA A BY TRANSPLANTING BONE MARROW CELLS

(75) Inventors: ASOK MUKHOPADHYAY, New Delhi (IN); NEELAM YADAV, New Delhi (IN)

(73) Assignee: NATIONAL INSTITUTE OF IMMUNOLOGY

(21) Appl. No.: 12/915,214

(22) Filed: Oct. 29, 2010

Related U.S. Application Data
(60) Provisional application No. 61/280,188, filed on Oct. 30, 2009.

(51) Int. Cl. A61K 35/12; A61P 7/00

(52) U.S. Cl. 424/93.7

(57) ABSTRACT

The transdifferentiation of bone marrow cells (BMCs) into hepatocytes can be used for the development of cellular medicine for degenerative and genetic diseases. Since the liver is the primary site of factor VIII (FVIII) synthesis, the partial replacement of mutated liver cells by healthy cells in hemophilia A (HA) could manage the severity of the bleeding disorder. The use of BMCs could be used as a therapy for the bleeding phenotype of hemophilia A and other related disorders.

### CROSS REFERENCE TO RELATED APPLICATION

This application claims the benefit of U.S. Provisional Application No. 61/280,188 filed Oct. 30, 2009 and claims the benefit thereof and incorporates the same by reference.

### BACKGROUND

Hemophilia A (HA) is an X-chromosome-linked recessive bleeding disorder with an incidence of 1 in 5,000 males. Severe HA patients have 1% or less of normal plasma factor VIII (FVIII) activity and spontaneously bleed. Patients with 1-5% of normal activity have less severe bleeding, and patients with 5-25% of normal activity usually bleed only with surgery or trauma. The clinical manifestation of this disorder is unpredictable, recurrent, and results in spontaneous bleeding in various parts of the body, including soft tissues, major joints and occasionally in internal organs. The standard treatment options for HA are either on-demand or prophylactic therapy with plasma derived or recombinant human FVIII. The therapeutic use of this purified factor can be a potential biohazard due to blood-borne pathogens, and may be ineffective due to the formation of inhibitors. Moreover, the life-long requirement for therapy can have a significant economic impact on patients.

Gene therapy has the potential to provide life-long correction of the bleeding disorder (Greengard J S, Jolly D J. Animal testing of retroviral-mediated gene therapy for factor VIII deficiency. Thromb Haemost. 1999; 82: 555-561; Park F, Ohashi K, Kay M A. Therapeutic levels of human factor VIII and IX using HIV-1 based lentiviral vectors in mouse liver. Blood. 2000; 96: 1173-1176; Chuah M K L, Schiedner G, Thorrez L, et al. Therapeutic factor VIII levels and negligible toxicity in mouse and dog models of hemophilia A following gene therapy with high-capacity adenoviral vectors. Blood. 2003; 101:1734-1743; Matsui H, Shibata M, Brown B, et al. Ex Vivo gene therapy for hemophilia A that enhances safe delivery and sustained In vivo factor VIII expression from lentivirally engineered endothelial progenitors. Stem Cells. 2007; 25: 2660-2669.)

Clinical trials using adeno-associated virus (AAV)-mediated factor IX have not conclusively shown long term therapeutic benefits in treating hemophilia B (HB) (Manno C S, Chew A J, Hutchison S, et al. AAV-mediated factor IX gene transfer to skeletal muscle in patients with severe hemophilia B. Blood. 2003; 101:2963-2972; Manno C S, Pierce G F, Arruda Y R, et al. Successful transduction of liver in hemophilia by AAV -Factor IX and limitations imposed by the host immune response. Nat Med. 2006; 12: 342-347.)

This may be due primarily to a cell mediated immune response against the adeno-associated virus (AAV) capsid protein that causes a decline in factor IX activity. Another concern associated with the use of viral vectors is the transient elevation of liver transaminases in response to the vectors. Therefore, alternate therapeutic options need to be developed.

Several tissues, like the spleen, lymph nodes, liver, and kidney have the potential to express the FVIII gene. While not being bound by any theory, some liver perfusion, and orthotopic liver transplantation studies in both animals and humans suggest that the primary site of FVIII synthesis is the liver and that within the liver, hepatocytes are the major FVIII producing cells.

Some studies have shown the therapeutic effectiveness of human hepatocytes transplanted under the kidney capsules of mice. (Ohashi K, Waugh I M, Dake M D, et al, Liver tissue engineering at extrahepatic sites in mice as a potential new therapy for genetic liver diseases. Hepatology. 2005; 41: 132-140.)

In addition to hepatocytes, sinusoidal endothelial cells have been shown as a cellular site of FVIII synthesis in mice. (Do H, Healey J F, Waller E K, Lollar P. Expression of factor VIII by murine liver sinusoidal endothelial cells. J Biol Chem. 1999; 274:19587-19592.)

A recent study demonstrated the therapeutic potential of liver sinusoidal endothelial cells (LSECs) in HA mice. (Follenzi A, Benten D, Novikoff P, Faulkner L, Raut S, Gupta S. Transplanted endothelial cells repopulate the liver endothelium and correct the phenotype of hemophilia A mice. J Clin Invest. 2008; 118: 935-945.)

Based on the above, either hepatocytes or LSECs could be candidate cells for the therapeutic intervention of HA, but these prior art treatments require cadavers or partially hepatectomised liver tissue from allogenic sources.

A cell-based therapy can become successful if the graft is easily obtained. Studies have demonstrated that bone marrow (BM) stem cells differentiate into hepatocytes in rodents. (Lagasse E, Connors H, AI-Dhalimy M, et al. Purified hematopoietic stem cells can differentiate into hepatocytes in vivo. Nature Med. 2000; 6:1229-1234; Wang X, Ge S, McNamara G, Hao Q L, Crooks G M, Nolta J A. Albumin-expressing hepatocyte-like cells develop in the livers of immune-deficient mice that received transplants of highly purified human hematopoietic stem cells. Blood. 2003; 10 1: 4201-4208; Jang Y, Collector M I, Baylin S B, Diehl A M, Sharkis S J. Hematopoietic stem cells convert into liver cells within days without fusion. Nature Cell Biol. 2004; 6:532-539; Khurana S, Mukhopadhyay A. Characterization of the potential subpopulation of bone marrow cells involved in the repair of injured liver tissue. Stem Cells. 2007; 25: 1439-1447; Khurana S, Mukhopadhyay A. In vitro transdifferentiation of adult hematopoietic stem cells: an alternate source of hepatocytes. J Hepatol. 2008; 49: 998-1007).

### SUMMARY OF THE INVENTION

Gene therapy can be used to treat hemophilia as the disease is caused by single gene defect and a small increase in gene products could essentially transform a severe form of hemophilia into a mild one. Cell-based therapies using isolated primary hepatocytes or LSECS can be used to treat clotting disorders.

Another aspect of the invention is the use of liver cells or hepatocytes derived from bone marrow cells in the phenotype correction of a subject with hemophilia HA.

Still another aspect of the invention is the use of liver cells or hepatocytes derived from bone marrow (BM) cells in managing bleeding disorders.

Other systems, methods, features and advantages of the present invention will be or become apparent to one with skill in the art upon examination of the following drawings and detailed description. It is intended that all such additional systems, methods, features and advantages be included within this description, be within the scope of the present invention, and be protected by the accompanying claims.

### BRIEF DESCRIPTION OF THE FIGURES

FIG. 1A shows the serum ALT level of male HA mice that were injected with acetaminophen.

FIG. 1B is a picture of a gel illustrating the induction of growth factor transcripts in liver tissue damaged by acetaminophen.

FIG. 1C shows tissue damage and regeneration of liver tissue.

FIG. 2A shows no engraftment of donor cells in the saline-injected control liver and engraftment of cells in the acetaminophen-treated liver.

FIG. 2B shows the presence of eGFP⁺ cells in the acetaminophen treated mice.

FIG. 2C shows the percentage increase of eGFP⁺ cells in the liver at different times after transplantation.

FIG. 2D shows immunohistochemical analysis of tissues of the heart, lung and kidney.

FIG. 2E shows absence of donor eGFP⁺ bone marrow cells in the bone marrow and spleen.

FIG. 3A shows that eGFP-expressing cells did not engraft in any of the sections examined from normal saline-injected mice.

FIG. 3B shows that in acetimophen-injected mice donor-derived (eGFP⁺) cells were present in all the sections examined, and expressed albumin, CK-18, and vWF

FIG. 3C shows that donor-derived (eGFP+) cells express both albumin (middle) and CK-18 (right) and shows nuclei of donor cells are not fused with recipient cells.

FIG. 3D shows that neither wild type nor HA mice liver tissue expressed CD45 antigen.

FIG. 4A shows that the number of albumin-expressing cells increased with time.

FIG. 4B shows expression of eGFP⁺ Albumin⁺ cells at different times.

FIG. 4C shows the percentage of eGFP⁺ cells that express vWF at different times.

FIG. 4D shows serum ALT, plasma creatinine and BUN levels of HAT mice compared with those levels in wild type mice.

FIG. 4E shows female and male control cells.

FIG. 4F shows a potential fused cell (XXXY) and an aneupolid cell (XXY).

FIG. 4G analysis of eGFP⁺ cells in the liver.

FIG. 4H shows a comparison between the binucleated cells (albumin-expressing) that are of recipient and donor-derived hepatocytes.

FIG. 4I shows a comparison between the binucleated cells (CK18-expressing) that are of the recipient and donor-derived hepatocytes.

FIG. 4J show liver and kidney of HAT mouse.

FIG. 5A shows isotype control in WT mice.

FIG. 5B shows FVIII-light chain (lC) staining in HA mice.

FIG. 5C shows FVIII-light chain staining in WT mice.

FIGS. 5D and E shows FVIII-light chain staining in HAT mice.

FIG. 6A shows whole blood clotting time in WT mice.

FIG. 6B shows the numbers of surviving and dead HAT mice after tail clip.

FIG. 6C shows primer pairs designed to amplify a sequence of the FVIII A3 domain.

FIG. 6 D shows the RT-PCR analysis for the synthesis of the target amplicon (537 bp).

FIG. 6E shows the RT-PCR analysis for the synthesis of the target amplicon (101 bp) in liver tissue samples and well as in the transplanted cells.

FIG. 6F shows real-time RT-PCR of the isolated hepatocytes and the endothelial cells from wild type and transplanted (HAT) mice.

FIG. 7 shows transmission electron microcopy images that FVIII (lc) protein is expressed in hepatocytes and endothelial cells of the wild type mouse liver, as well as in the bone marrow-derived hepatocytes and endothelial cells in transplanted mice.

FIG. 8A shows FVIII antigen expression in HAT mice.

FIG. 8B shows relative FVIII activity in HAT mice.

FIG. 8C shows a standard curve of normal pooled plasma FVIII activity versus clotting time in seconds.

FIG. 8D shows the aPTT values of pooled human and WT, HA, HAT mice.

FIG. 8E shows relative FVIII activity.

FIG. 8F shows FVIII inhibitors in HAT mice and patients plasma.

FIG. 9A shows results of clotting time in WT, K/O and HAT mice

FIG. 9B shows results of tail clip challenge in WT, K/O and HAT mice

FIG. 9C shows the relative FVIII activity in K/O and HAT mice as determined by Coatest.

FIG. 9D shows relative plasma FVIII activity in WT, K/O and HAT mice as determined by aPTT.

FIG. 10A shows confocal images taken five months after transplantation, mice were sacrificed and liver cryosections were analyzed by. IHC. Five-micron serial sections were stained with anti-GFP, anti-vWF, and anti-FVIII antibodies. (A) Confocal images. The merged picture shows that donor-derived (eGFP) cells express vWF.

FIG. 10B shows that donor-derived (eGFP) cells express FVIII light chain protein.

FIG. 10C shows that vWF expressing (endothelial) cells express FVIII light chain protein.

### DETAILED DESCRIPTION OF THE INVENTION

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein.

The inventors have demonstrated that BMCs can be differentiated into hepatocytes and endothelial cells in vivo, Hematopoietic stem cells (HSCs) can differentiate into hepatocytes and endothelial cells in the presence of damaged liver tissue. The inventors have shown that acetaminophen at high doses or use of monocrotalin causes centrilobular necrosis, and, in the experiments discussed below, it was used to induce acute liver injury. While not being bound by any theory, it is surmised that the toxicity of acetaminophen is due to its highly reactive metabolic product N-acetyl-p-benzoquinone imine, which prevents Ca₂⁺ homeostasis inside cells by oxidizing thiol groups on proteins.

As described in more detail below, the host liver was perturbed with acetaminophen or monocrotalin to facilitate the engraftment and hepatic differentiation of lineage-depleted (Lin) enhanced green fluorescent protein (eGFP)-expressing BMCs. BMCs were transplanted into HA mice. There was a gradual increase in donor-derived hepatocytes suggesting that there were several rounds of division in engrafted cells. HGF and TGF-α, two known strong hepatic mitogens, synthesized by the damaged liver tissue, might have triggered cellular proliferation. In contrast, BM-derived LSECs were much lower in number. Immunochemistry experiments with the liver tissue showed that the donor-derived cells expressed the markers of both hepatocytes (albumin and cytokeratin-18) and endothelial cells (von Willbrand factor). The results of fluorescent in situ hybridization and immunocytochemistry experiments suggested that differentiation was direct in this model.

The transplantation study showed that BMCs not only differentiated into hepatic and liver sinusoidal endothelial cells, but also that they expressed the intact gene of the FVIII A3 domain, which was disrupted in HA mice. The IHC results confirmed that HAT mice synthesized the light chain FVIII, which is missing in HA mice. The BMC-recipient mice expressed FVIII protein and survived in a tail clip challenge experiment. Furthermore, a coagulation assay confirmed that the plasma FVIII activity was maintained at 20.4±3.6% of normal pooled plasma activity for more than a year without forming its inhibitor. Flow cytometric and IHC results combined with the morphology of the cells expressing the FVIII light chain protein suggested that the missing factor was produced by hepatocytes. Orthotopic liver transplantation studies in both animals and humans suggest that the primary site of FVIII synthesis is the liver. Within the liver, hepatocytes were found to be the major source of FVIII.

The inventors presume that FVIII was produced in the liver, as donor cells were not detected in other organs such as the heart, lung, etc. The absence of donor cells in these organs, specifically in bone marrow, was because non-irradiated mice were used in this investigation. eGFP+ cells were not found in the peripheral blood. The results do not exclude the possibility of LSECS as source of FVIII.

It was shown that transplanting human hepatocytes under the mouse kidney capsule elicits therapeutic effect through the secretion of FVIII. Recently, a therapeutic effect of hepatocytes in HB mice has been documented. Tatsumi K, Ohashi K, Shima M, Nakajima Y, Okano T, Yoshioka A. Therapeutic effects of hepatocyte transplantation on hemophilia B. Transplantation. 2008 15; 86:167-170.

The transplantation of BMCs into HA mice restored plasma FVIII activity well above the therapeutic correction level, which could eliminate spontaneous bleeding. Moreover, during the 18 month investigation the formation of FVIII inhibitors was not observed.

The BMCs used in the method of this invention can be any cells derived taken from or derived from bone marrow such as mononuclear cells, Lin⁻ cells, mesenchymal stem cells, hematopoietic cells. The number of cells injected at one time will depend on the type of cells and the condition and health of the subject. In the mice model described below 0.1 to 1×10⁶ Lin⁻ BMC cells/mice are injected. Cells can be administered in single or multiple doses and 0.1 to 1×10⁶ Lin⁻ BMC cells can be injected in a single dose or multiple doses. The cells can be administered intravenously, intraspleenic or through the porter vein. In the case of intraspleenic and porter vein routes, mice have to be conditioned by injection with FVIII to increase its level above 20% of the normal mouse FVIII level.

The inventors have also tried monocrotaline induced liver damage model and it damages the endothelial cells as well as hepatocytes. Transplantation of Lin⁻ BMC also showed phenotype conversion in monocrotaline induced liver damage model. ELISA and COATEST assays showed the expression of FVIII in the plasma.

BMC transplanted mice may be treated with growth factors like HGF and EGF to improve the outcome.

Thus, the inventors have established that BMC therapy in HA mice (liver injury model) results in phenotypic rescue and reduces mortality due to blood loss and it is expected that this result will be reproduced in humans. From the studies described below, it can be concluded that BMC therapy might be an effective way to control the bleeding disorder in subjects with HA.

Uncommitted bone marrow cells can be used to correct a bleeding disorder in a subject with HA including a human. In case of normal (undamaged) liver, BMCs are not expected to be differentiated into hepatocytes. In that case, in vitro differentiated hepatocytes/liver cells should be used as the graft. (Khurana and Mukhopadhyay, J. Hepatol. 49, 998-1007, 2008). In the case of human subjects, since one should not damage liver, prior to transplantation the crude or purified BMC will be differentiated in vitro into hepatocytes by using specialized culture medium. The hepatocytes can be produced by transdifferentiation of HLA matched BM cells in the culture. Once BMC differentiate into hepatocyes, it is expected that liver engraftment will be normal as in the case of primary hepatocytes. These hepatocytes will then be administered to a patient in an amount sufficient to express Factor VIII. One measure of treatment is a decrease in coagulation time.

The number of hepatocytes transplanted must be sufficient to change the phenotype initially or after division in vivo. In case of human subject, about 20 to 25×10⁶ differentiated hepatocytes would be required for transplantation through the portal vein or systematic delivery, although this number could vary. Multiple dosages of cells supported by hepatocyte growth factor (HGF) injection will improve the engraftability of the cells and faster recovery. Cells are administered in a media that is pharmaceutically acceptable such as normal saline.

While in the case of human subject, the liver should not be damaged intentionally it is possible that if the subject's liver is damaged, BMCs such as HLA matched donor Lin-depleted BM cells can be transplanted directly in the patient. These cells after engraftment in the liver may transdifferentiate into hepatocytes and endothelial cells. In this embodiment about 10 to 25×10⁷ cells would be transplanted in the same manner as described above.

BMCs are shown to have a tendency to fuse with primary hepatocytes in vivo to attain the hepatic phenotype. Cell fusion occurs during normal mammalian development. In adult mammals, cell fusion may be associated with chromosomal abnormalities that can lead to oncogenic transformations. Thus, it is crucial to investigate the fusion between donor and recipient cells, if any. In the liver damage model, we and other groups have already shown that BMCs can directly differentiate into hepatocytes. The results of FISH experiments suggested that, like in previous reports, cell fusion unlikely occurred in the present model of the phenotypic rescue of HA mice. In other words, these results illustrate the possibility of the direct differentiation of BMCs into functional liver cells.

Like many other treatments that are used for the treatment of various conditions, this treatment may not be effective for every patient. Factors that may influence or determine the effectiveness of the treatment may include and are not limited to the extent of any liver damage in the patient and the cause of the liver damage may have an effect in whether BM-derived cells will fuse with host cells or will directly differentiate into hepatocytes.

The application of BM-derived hepatocytes in managing HA has distinct advantages over alternative approaches:

(i) HLA-matched BMCs can be easily obtained through a bone marrow donor registry,

(ii) there is no need to identify the effective sources of graft as in the case of LSECs, and

(iii) VIII produced by BM-derived cells is structurally and functionally identical to the native molecule, and

(iv) a lower amount of FVIII inhibitor is formed, which will make this therapy successful for the sustained correction of hemophilia A.

The tolerogenic properties of the liver seem to have an important role in maintaining the synthesis of FVIII by donor cells for a longer duration.

The methods described above can also be used to treat metabolic diseases of the liver, alpha 1-antitrypsin (A1AT) deficiency, von Willebrand disease, and coagulation disorder like hemophilia B.

Unless, otherwise indicated, all numbers expressing quantities of ingredients, reaction conditions, and so forth used in the specification and claims are to be understood as being modified in all instances by the term “about”. The terms “a”, “an” and “the” refer to “one or more” when used in this application, including the claims.

Abbreviations used herein include:

ALT—alanine aminotransferase

aPTT—activated partial thromboplastin

A1AT—alpha 1-antitrypsin

BM—bone marrow

BMC—bone marrow cell

FISH—fluorescence in situ hybridization

HA—hemophilia A

HAT—hemophilia A transplanted

HB—hemophilia B

HGF—hepatocyte growth factor

HSC—Hematopoietic stem cells

IHC—immunohistochemistry

LSEC—liver sinusoidal endothelial cell

TGFα—transforming growth factor α

TEM—transmission electron microscope

vWF—von Willbrand factor

The following examples provide illustrative embodiments. In the light of the present disclosure and the general level of skill in the art, those of skill will appreciate that the following examples are intended to be exemplary only and that numerous changes, modifications, and alterations can be employed without departing from the scope of the presently claimed subject matter.

### Example 1

The techniques of this example were used in the Examples described below.

Animals: Six to eight-weeks old male HA mice [B6; 129S4_FstmlKaz/J] and eGFP expressing [C57BL/6-Tg(UBC-GFP)30Scha/J] female mice were used in this investigation. Mice were obtained from The Jackson Laboratories (Bar Harbor, Me.) and maintained in the National Institute of Immunology's experimental animal facility. Mice were kept in an isolator, fed with autoclaved acidified water and irradiated food ad libitum. All experiments were conducted as per procedures approved by the Institutional Animal Ethics Committee.

Acute liver injury model: Acute liver injury was induced by the intraperitoneal injection of acetaminophen (500 mg/kg b.w.) in male HA mice. Animals were sacrificed at days 1, 2, or 3 after the administration of acetaminophen, and serum samples were collected for the analysis of alanine aminotransferase (ALT) level. The liver was dissected and fixed in 10% buffered formalin. Five-micron paraffin-embedded tissue sections were stained with Hematoxylin and Eosin for histological examination. The liver functions of control (untransplanted) and transplanted mice were determined by ALT, and renal functions by creatinine and blood urea nitrogen (BUN) analyses. These assays were carried out using standard kits (Transasia BioMedicals, Daman, India).

Liver reconstitution: Lin⁻ (CD5, CD11b, CD45R, 7-4, Gr-1 and Ter119 depleted) BMCs were isolated from eGFP transgenic female mice by a magnetic cell sorter (Miltenyi Biotech., Gladbach, Germany) following a negative selection method. Two-hundred-and-fifty-thousand sorted cells were transplanted in acetaminophen-treated (1 day) and control HA male mice through the tail vein.

Flow cytometry: The antibodies and conjugates used for the flow cytometric analysis were goat anti-mouse albumin (A90-134A) at 1:200 (Bethyl, Montgomery, Tex.), goat anti-von Willebrand factor (vWF) (sc-8086) at 1:100 (Santa Cruz Biotechnology, Santa Cruz, Calif.) and anti-goat IgG phycoerythrin (PE) (Jackson Immunologicals, West Grove, Pa.). Prior to staining, single cells from the liver were obtained by a two-step enzymatic method. (Wang Y J, Li M D, Wang Y M, Nie Q H, Chen G Z. Experimental study of bioartificial liver with cultured human liver cells. World J. Gastroenteroi. 1999; 5:135-137.) Cells were fixed in 4% paraformaldehyde and permeabilized with 0.1% saponin in 1% bovine serum albumin-PBS for 30 min at 4° C. The antibody-labeled cells were analyzed with a flowcytometer (BD-LSR, BD Biosciences, San Jose, Calif.).

Immunohistochemistry (IHC): Liver tissues were fixed in 4% paraformaldehyde, cryoprotected in 30% sucrose solution for 24 h at 4° C., and frozen in tissue freezing medium. Five-micron serial sections were treated with 0.15% Triton X-100 for 30 min at room temperature. The sections were then stained with anti-GFP, anti-mouse albumin (A90-134A) (Bethyl, Montgomery, Tex.), anti-CK-18 (sc-32329) and anti-von Willbrand factor (vWF) (sc-8086) (Santa Cruz Biotechnology, Santa Cruz, Calif.) antibodies for 1 h at room temperature. The secondary antibodies were conjugated with AlexaFluor 488/594 (Molecular Probes Inc., OR).

The nuclei were stained with 4′,6-Diamidino-2-phenylindole (DAPI). Sections were imaged under an Olympus fluorescence microscope and a DP70 digital camera. The Olysia BioReport software was used for the image acquisition. The images were composed and edited in Photoshop 6.0 (Adobe).

Isolation of liver cells: Single cells from the liver were obtained by a two-step enzymatic method. (Wang Y J, Li M D, Wang Y M, Nie Q H, Chen G Z. Experimental study of bioartificial liver with cultured human liver cells. World J. Gastroenteroid. 1999; 5:135-137). In brief, mice were anesthetized by intraperitoneal injection of ketamine (100 mg/kg b.w) and xylazine (15 mg/kg b.w). In the first step, the liver was perfused with Ca²⁺/Mg²⁺ ion-free Hank's buffer (50 ml) for 10-15 min. In second step, 50 ml of collagenase IV (Sigma, St. Louise) solution (0.3 mg/ml) was used for perfusion. The liver capsule was incised and single cell suspension was prepared in RPMI-1640 medium. The viability of cells was determined with trypan blue dye exclusion method.

Immunostaining of FVIII: The tissue samples were fixed in 10% buffered formalin, embedded in paraffin and sectioned (5μ). Five-micron deparaffinized sections were treated with 0.3% H₂O₂ to inactivate endogenous peroxidase. The sections were separately stained with anti-FVIII light chain (sc-33584, Santa Cruz) specific antibody and a corresponding isotype control serum (S-5000, Vector Laboratories, Burlingame, Calif.), followed by anti-rabbit IgG-HRP. Finally, the sections were reacted with 3,3′-diaminobenzidine (DAB) substrate (Vector Laboratories), washed and counter stained with Mayer's hematoxylin. Sections were imaged using bright field microscopy.

Sex chromosome fluorescence in situ hybridization (FISH) and Immunocytochemistry: The same liver cells of HAT mice were used for FISH and immunocytochemistry. The interphase FISH study was performed using whole-chromosome paint probes specific for chromosomes X and Y (RTU Mouse WCP FITC chromosome X and RTU Mouse WCP Cy3 chromosome Y; Cambio Ltd., Cambridge, UK). The cells were treated in a hypotonic (50 mMol KCl) solution for 30 min and fixed in a methanol and acetic acid mixture (3:1 ratio). A 25-microliter cell suspension was used to prepare each slide. The slides were treated with 70% acetic acid for 1 min and dehydrated in increasing concentrations of alcohol (70%, 90% and 100%). The cells were further treated in pepsin solution (10 mg/l, pH 2.0) at 37° C. for 20 min, washed in PBS, further fixed in 1% paraformaldehyde at 4° C. for 10 min and finally dehydrated as before. Ten microliters of each labeled probe were used for FISH. Probes and nuclear DNA together were denatured at 75° C. for 5 min in hybrite (Vysis Inc., Downers Grove, Ill.) and incubated in a humidified chamber at 37° C. for 18 h. Slides were washed using 0.03% and 0.01% NP40 at 72° C. for 2 min each. The specimens were dehydrated in alcohol and mounted with antifade medium containing DAPI. The FISH analysis was carried out using an Olympus BX51 microscope with an epifluorescence attachment and images were captured through the spectral imaging system.

For immunocytochemical analysis, cytospins were fixed and permeabilized as before. The cells were stained for albumin and CK-18, and examined under an Olympus fluorescence microscope.

Phenotypic correction: Phenotypic correction was assessed by the tail clip challenge test, as described in the literature. (Connelly S, Andrews J, Gallo A. Sustained phenotypic correction of murine hemophilia A by in vivo gene therapy. Blood. 1998; 91: 3273-3281). Tails of the transplanted mice were clipped at a length of 1.5 cm, without subsequent cauterization. Three groups of mice were subjected to tail clip challenge: wild type (positive control), HA (negative control), and HA transplanted (HAT) test mice. The clotting of blood and subsequent survival of mice was used to indicate the normal or corrected HA phenotype. The surviving mice were maintained for 3-6 months (mo) for additional studies.

In another assay, the whole blood clotting times of the above three mice groups were determined. In brief, blood was withdrawn in a capillary tube (nonheparinized) from the eye. The loaded capillary tube was gently broken into two-halves, slowly pulled the ends apart to view the insoluble fibrin strands.

FVIII assays: The FVIII protein was measured using ELISA. The assay was performed on the basis of a published protocol, (Follenzi A, Benten D, Novikoff P, Faulkner L, Raut S, Gupta S. Transplanted endothelial cells repopulate the liver endothelium and correct the phenotype of hemophilia A mice. J Clin Invest. 2008; 118: 935-945) with minor modifications. In brief, 96-well microtiter plates were coated with 100 μl of diluted mouse plasma (1:160) in carbonate/bicarbonate buffer by incubating at 4° C. for overnight. Plates were washed with PBS containing 0.05% Tween 20 (PBST). The wells were blocked with 200 μl of 5% horse serum in PBST for 1 h and then reacted with 100 μl of rabbit anti-FVIII polyclonal antibody (ab53703; Abcam, Cambridge, UK) at 1:3000 dilution. The plates were incubated at 37° C. for 2 h, washed and then further reacted with 100 μl goat anti-rabbit IgG-HRP (1:1000) at 37° C. for 1 h. Again, the plates were washed, 100 μl of 3,3′,5,5′-tetramethylbenzidine (TMB) substrate (BD OptEIA™, BD Biosciences) was added in each well and incubated at 37° C. in the dark for 15 min. The reaction was stopped with 50 μl of 2NH₂SO₄, and the plates were read at 450 nm on a PowerWave XS (BioTek Instuments Inc., Winooski, Vt.) reader. The amount of FVIII antigen in HAT plasma was determined relative to WT and HA plasma run simultaneously.

COATEST assay: FVIII activities in the plasma samples of WT, HA and HAT mice were analyzed according to the manufacturer's instructions.

The amount of FVIII antigen in HAT plasma was determined relative to plasma from WT and HA mice run simultaneously. The FVIII activity was measured using COATEST FVIII kit (Instrument Laboratory Company, Lexington, Mass.).

Coagulation assay: The FVIII assay was performed as described in the literature with minor modifications. Blood samples were collected in microfuge tubes containing sodium citrate. The samples were centrifuged and the plasma fractions were immediately stored at −70° C. The activated partial thromboplastin time (aPTT) assay was performed by incubating 100 μl of a PTT reagent (Diagnostica Stago, Asnieres, France) with 100 μl of plasma at 37° C. for 5 min. Then, 100 μl of 25 mM CaCl₂ was added and the clotting time was determined. To establish the standard curve, pooled mouse and human plasma (separately) at dilutions of 1:10, 1:50 and 1:250 in Owren-Koller buffer was subjected to an aPTT assay. The clotting times of the diluted plasma were considered equivalent to that of 100%, 20% and 4% normal plasma, respectively. For the test mice, 100 μl of diluted (1:10) mouse plasma, 100 μl of hFVIII-deficient plasma, and 100 μl of a PIT reagent were used, which was followed by 100 μl of CaCl₂. For the human samples, the mouse plasma was replaced with human plasma. The plasma FVIII activities with respect to normal pooled plasma were determined by comparing clotting times from the standard curve.

FVIII inhibitor evaluation: The FVIII inhibitor was measured using a Bethesda assay and the results were expressed as Bethesda Unit/ml (BU/ml). One Bethesda Unit is defined as the reciprocal of the dilution of test plasma that inhibits 50% of the total FVIII activity after 2 h of incubation at 37° C. For the standard curve, 200 of normal pooled mouse/human plasma and 200 μl of buffer were incubated at 37° C. for 2 h. For the samples, 200 μl of normal pooled mouse/human plasma were incubated with 200 μl of undiluted and diluted (1:1 to 1:64) test plasma at 37° C. for 2 h. The samples were further diluted to 1:10, 1:50 and 1:250 before the FVIII activity assay. The dilution of the sample at which 50% inhibition occurred was determined.

RT-PCR and real-time RT-PCR: Details of the total RNA extraction from whole liver tissue and isolated cells, and cDNA synthesis are provided in Supplementary methods. cDNA synthesized from the whole liver or hepatocytes/LSECs of WT mice were considered as positive controls, while cDNA synthesized from the whole liver of HA mice or Lin⁻ WT BMCs was considered as a negative control. Primer pairs were designed using Primer 3 software (for RT-PCR) and Primer Express® software version 2.0 (Applied Biosystems, Foster City, Calif., USA) for real-time RT-PCR. The sequences of the primers and amplification conditions of the reactions are shown in the Table 1A & 1B. Real-time RT-PCR was performed with the Master Cycler ep Realplex 4 (Eppendorf A G, Hamburg, Germany) according to the manufacturer's instructions. Real-time RT-PCR was carried out in quadruplicates of each 10 μl reaction volume containing 5 μl 2× Power SYBR® Green PCR Master Mix (Applied Biosystems), 0.5 μM of each primer, and 1 μl of cDNA. For normalization, the expression level of the housekeeping gene (GAPDH) was measured as an endogenous control. For quantification of each PCR result, we calculated the ΔCt value between the target gene of each cell type and its endogenous control. ΔCt values of hepatocytes or endothelial cells were subtracted from the ΔCt value of HA whole liver or Lin⁻ BMCs to obtain ΔΔCt. The 2^{-ΔΔCt} value (relative) was calculated using Realplex 2.2 software (Eppendorf) and expressed as log fold change.

Transmission electron microscopy (TEM): Immunogold labeling of tissue sections was performed according to methods published in the literature [24]. In brief, ultra-thin (70 nm) tissue sections were taken on nickel grids. The sections were initially blocked with 2% skimmed milk and then incubated separately on droplets of a 1:50 dilution anti-albumin or anti-vWF antibodies for 4-6 h at 6-8° C. After washing the sections with PBS containing 1% fish gelatin (6×2 min), we further incubated the sections for 2 h with anti-goat IgG conjugated to 10 nm colloidal gold particles. The sections were thoroughly rinsed with PBS containing 1% fish gelatin (6×2 min). In the second step of immunolabeling, all sections were incubated on droplets of anti-FVIII antibody (1:50) for 4 h at 6-8° C. Washed sections were incubated with anti-rabbit IgG conjugated to 40 nm colloidal gold particles (1:20) for 2 h. The washed sections were stained with uranyl acetate and lead citrate before microscopic analysis (Morgagni 268D, FEI, Netherlands). Except for primary antibodies, all other reagents were procured from TAAB Laboratories Equipment Ltd. (Berkshire, England).

Statistical values: The results of multiple experiments were reported as the mean±Standard Error Mean (SEM). Tukey-Kramer multiple comparison tests were then used to compare between two means.

### Example 2

### Acetaminophen-Induced Acute Liver Injury in HA Mice

Acute liver injury was induced by the intraperitoneal injection of acetaminophen into HA mice as described above.

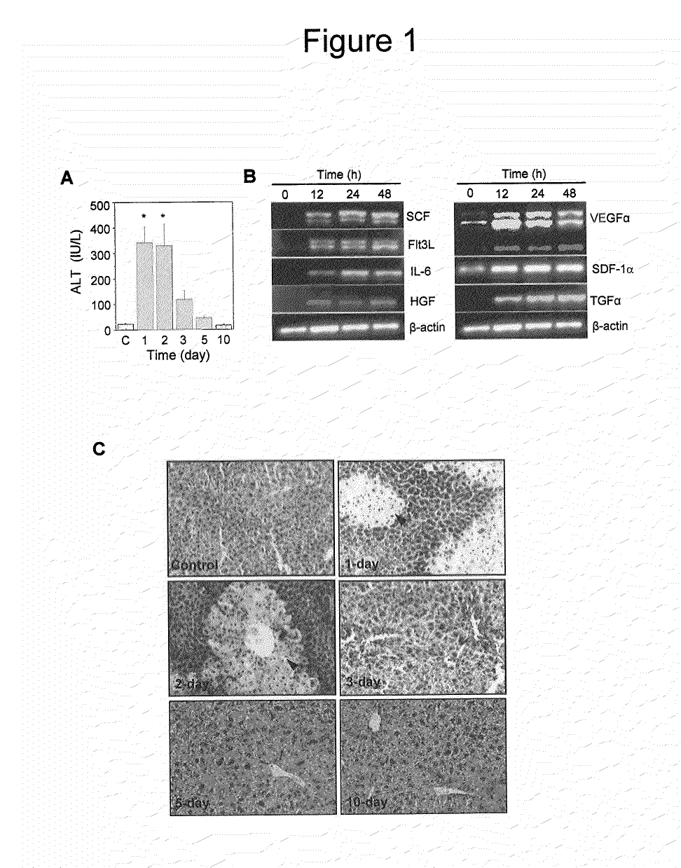

FIGS. 1A, 1B and 1C show the effect of the acetaminophen treatment on liver function and cytokine gene expression. Acetaminophen was administered to male HA mice by an intraperitonial injection. The control mice received saline. At each time interval, the serum ALT level was estimated. Control mice ‘C’ received normal saline (n=6 for each group, *p<0.001) and these results are shown in FIG. 1 A. A significant increase in the serum ALT level was observed within one day of the acetaminophen injection compared to the control animals. The ALT level was high up to the second day of treatment, after that it slowly normalized with time.

The effect of the acetaminophen on the liver pathology was examined. Representative H&E stained sections of control and acetaminophen treated liver at different times are shown (FIG. 1C) The liver of the control animal was histologically normal in its lobular architecture. Tissue necrosis was detected within a day of acetaminophen administration. Tissue damage was primarily localized near the central vein (black arrow). A histopathological examination revealed hemorrhagic centrilobular necrosis in the hepatic tissue on the first and second days of [after]acetaminophen treatment. The necrotic liver started regeneration after the third day of treatment. (FIGS. 1A and 1C) The liver pathology became normal from the 5^{th} day [after] of acetaminophen treatment. (Magnification 40×). Since the liver was completely restored within 10 days of the acetimophen treatment, we predicted that the tissue microenvironment induced its regeneration.

Acetaminophen was administered to several mice and they were sacrificed at 12, 24 and 48 h. The control mice received saline. RT-PCR analysis was used to analyze the gene expression of several growth factors known to be involved in hematopoietic and hepatic activities of different growth factor transcripts and this is shown in FIG. 1B. The results indicated no expression of the stem cell factor (SCF), fetal liver tyrosine kinase-3 (Flt-3) ligand, interleukin-6 (IL-6), hepatocyte growth factor (HGF), and transforming growth factor-α (TGF-α) genes in the control liver tissue. (FIG. 1B) However, their expression was upregulated within 12 h of the acetaminophen treatment. The expression of the vascular endothelial growth factor α (VEGFα) gene also increased in the damaged tissue, though basal level expression was observed in the control tissue. (FIG. 1B)

### Lin⁻GFP⁺ BMCs Engrafted in Damaged HA Mice Liver

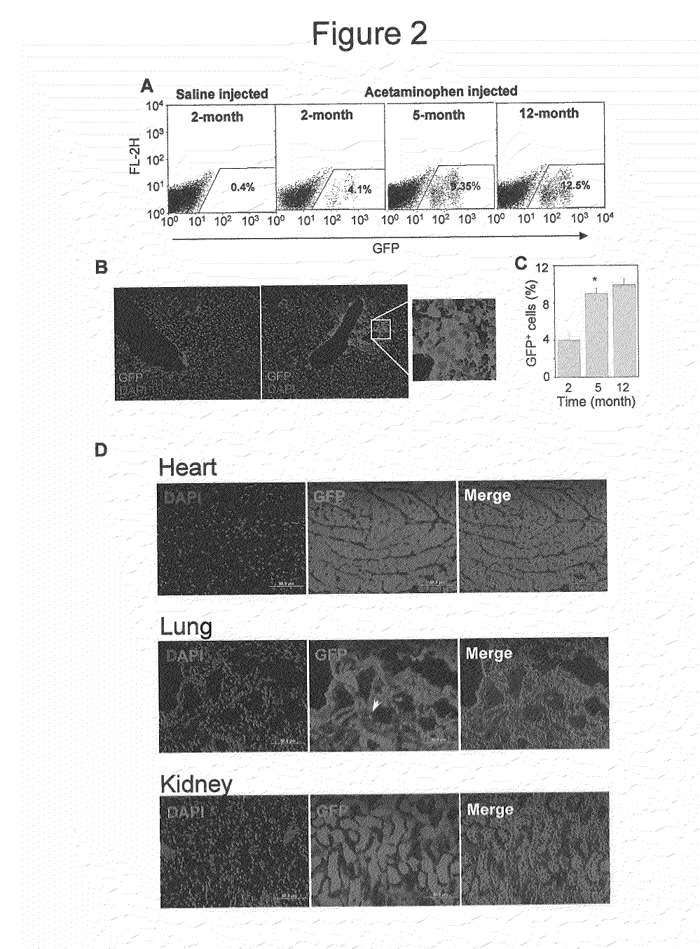

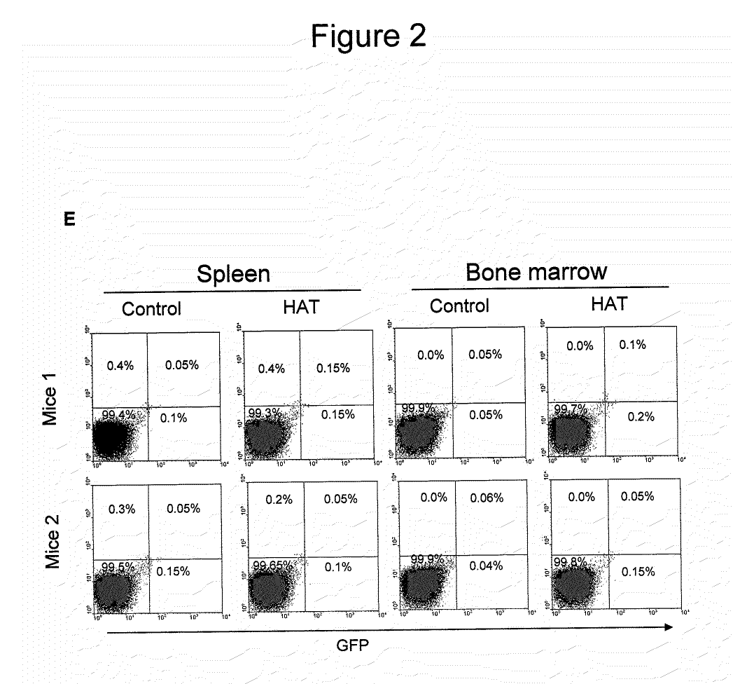

Two-hundred-fifty-thousand Lin⁻ cells were transplanted in HA mice 24 hours after the administration of normal saline (control) and acetaminophen. The number of engrafted cells (eGFP⁺ cells) in the recipient liver was determined by flow cytometric analysis. Representative dot-plots showed no engraftment of donor cells in the saline-injected control liver, whereas the cells were engrafted in the acetaminophen-treated liver. Representative dot-plots show an increase in eGFP⁺ cells at different times after transplantation in acetaminophen-injected mice (FIG. 2A). IHC analysis of liver sections of saline (left) and acetaminophen (right) injected mice after 5 mo of transplantation are shown in FIG. 2B. A representative photomicrograph (FIG. 2B) shows the presence of eGFP+ cells near the central vein of acetaminophen injected mice. The inset indicates the magnified region of the section (magnification 20×). The percentage increase of eGFP⁺ cells in the whole liver at different times of transplantation. mo: month, n=4 for each time point, *p<0.05 is shown in FIG. 2C. The damaged liver tissue expressed the SDF-1α transcript (FIG. 1B), the product of which is known to direct the migration of CXCR4-expressing BMCs. In the acetaminophen-treated mice, the donor cells were typically detected near the central vein, where severe tissue necrosis was observed (FIGS. 2 A and 2 C, right). The number of donor-derived cells progressively increased in the HAT mice, and about 10.2±2.3% eGFP⁺-cells were detected in the recipient liver 12 mo after transplantation (FIGS. 2A and 2C).

The bio-distribution of donor cells in different organs, such as the heart, lung, kidney, spleen and bone marrow was examined. Five months after transplantation, the mice were sacrificed and serial sections of heart, lung and kidney were stained with anti-GFP antibody. Immunohistochemical analysis of serial tissue sections of the heart, lung and kidney suggested that eGFP⁺ BMCs do not engraft in these organs (FIG. 2D) Representative images show no engraftment of donor cells in these organs (white arrow indicates non-specific stain). Number of mice examined (n)=2. Magnification 20×. The spleen and bone marrow of the above mice were analyzed by flow cytometry for the presence of eGFP⁺ cells. Surprisingly, flow cytometric analysis of BM and spleen also revealed the absence of cells (eGFP⁺) in these tissues (FIG. 2E) and this shows that neither eGFP nor CD45 expressing cells are present in the control liver section. (n=2).

### Engrafted Cells Differentiate into Hepatic and Endothelial Lineage

IHC and FISH analyses of HAT mice.

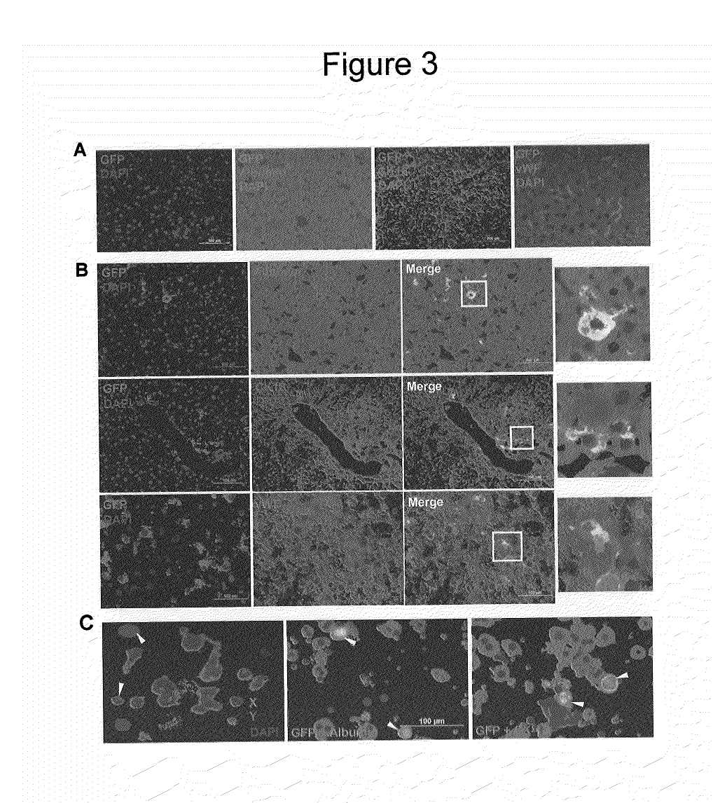

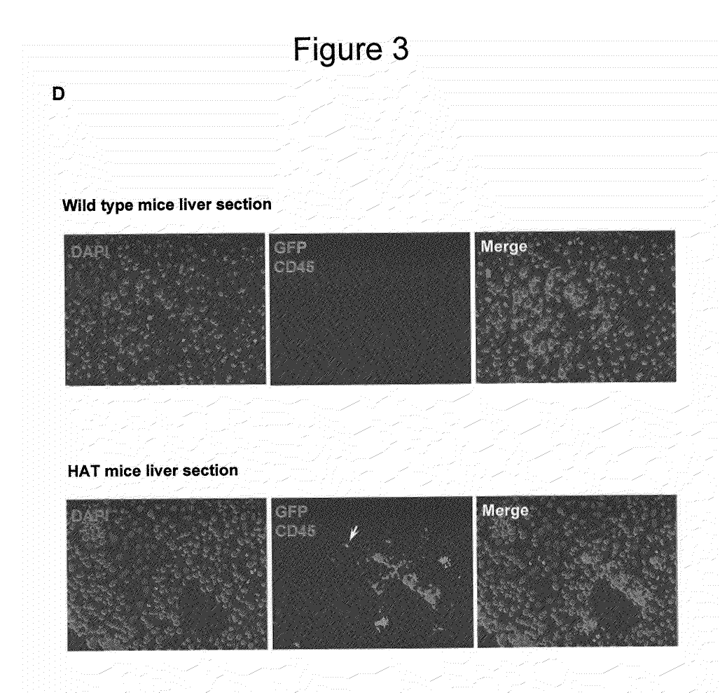

One day after normal saline (control) and acetaminophen administration, each mouse received a tail vein injection of donor cells. To determine whether engrafted cells differentiate into the target tissue, serial liver sections by IHC were analyzed. Two months after transplantation, mice were sacrificed and liver cryosections were analyzed by IHC. Five-micron serial sections were stained with anti-GFP, anti-albumin, anti-CK 18, and anti-vWF antibodies. The representative micrographs show that eGFP+ cells did not engraft in the control mice (injected with saline), and albumin, CK-18, and vWF were normally expressed in the host tissue (FIGS. 3 A). IHC analyses of acetaminophen-treated mice not only revealed that the donor-derived cells engrafted in the liver, but additionally showed that the eGFP⁺ cells were also expressing albumin (top panel), CK-18 (middle panel) and vWF (bottom panel) (FIG. 3B). (n=4). The insets indicate the magnified region of the tissue sections. As secondary antibody controls, the sections were also stained with respective conjugates (data not shown).

In order to check for the expression of hematopoietic marker in the engrafted cells, tissue sections were co-stained with eGFP and CD45 antibodies. It was found that neither wild type nor HAT mice liver tissue expressed the CD45 antigen. Neither eGFP nor CD45 expressing cells are present in the control liver section (FIG. 3D) (top panel). In HAT mice eGFP⁺ cells are present but CD45 antigen is not expressed by the cells (bottom panel). White arrows indicates non-specific staining. These results suggest that the donor cells might have differentiated into hepatocytes and endothelial-like cells.

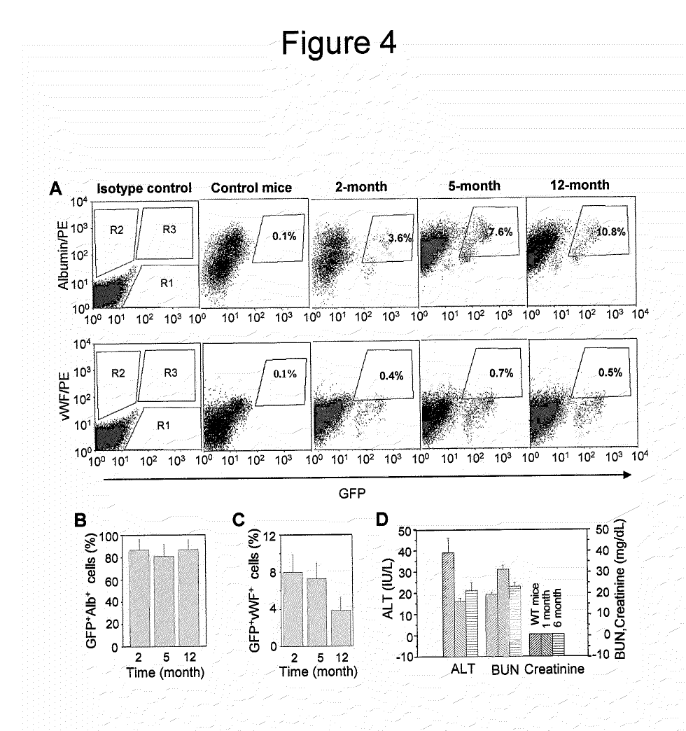

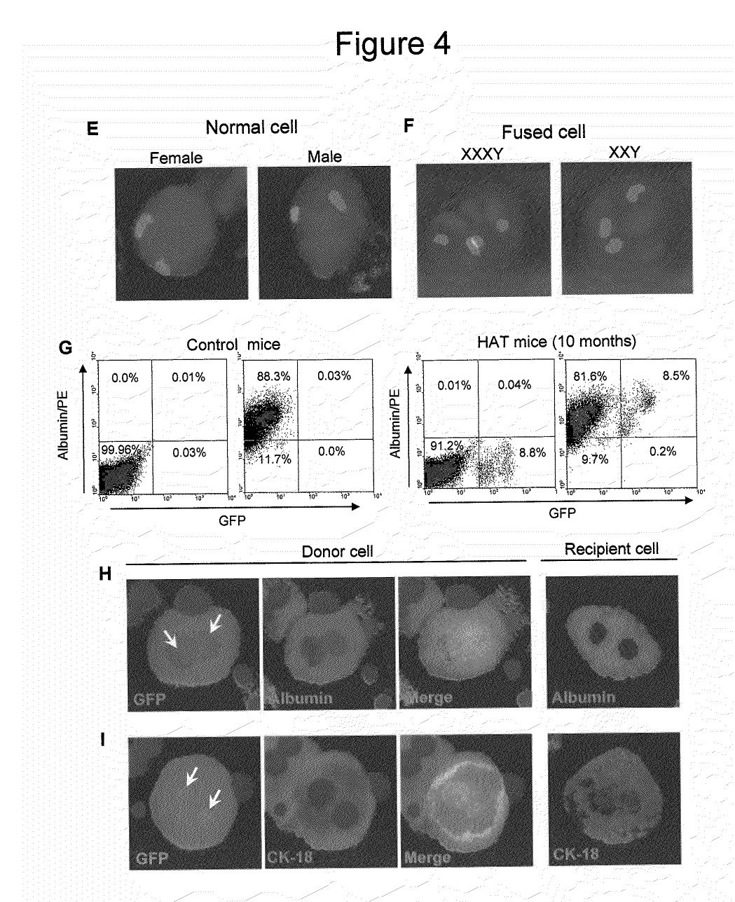

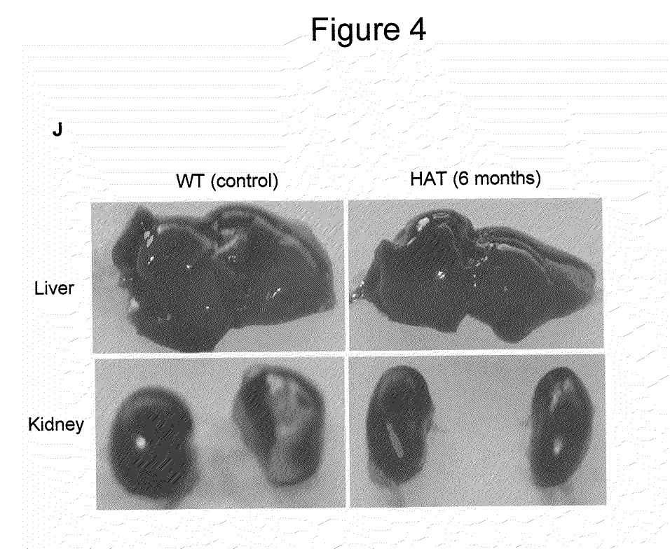

Sex chromosome FISH and immunocytochemistry were also conducted. To study the possibility of fusion between donor (XX) and recipient (XY) cells, nine slides from three HAT mice were examined by FISH. Prior to examining the fusion between donor and recipient cells, the specificity of the XX and XY probes on a bone marrow metaphase preparation was evaluated. Female and male bone marrow cells were separately tested for the specificity of probes by adding mixed probes. Representative images show that the probes are specific as Y chromosome (Cy3) was not detected in the female control cells and only one X chromosome (FITC) was detected in male control cells. (FIG. 4E), indicating that the FISH probes were specific. The cytogenetic analysis of 4454 nuclei suggests that discrete donor and recipient cells made up 9.45% and 90.35% of the tissue, respectively (Table 2). Furthermore, the fraction of cells containing XX and XY chromosomes was 0.2%, indicating that the probability of fusion between donor and recipient cells was negligibly low (Table 2).

Liver cells of HAT mice (10 mo after transplantation) were labeled with X (FITC) and Y (Cy3) chromosome-specific probes. The fusions between donor (XX) and recipient (XY) cells were microscopically examined and this is shown in FIG. 3(C). White arrows are used to indicate donor-derived unfused cells (left) panel.

A representative micrograph of interphase FISH of the HAT mice liver cells show two distinct nuclei of donor cells (XX), which are not fused with the recipient cells (FIG. 3C, left).

Representative images show a fused cell (XXXY) and aneuploid (XXY) cells. A potential fused cell (XXXY) and an aneuploid cell (XXY) are shown in FIG. 4F. In order to prove that the eGFP+ cells present in the liver are hepatocytes, we separately analyzed the same cells by immunochemistry. The same liver cells were immunostained for albumin and CK-18. It was observed that, like recipient hepatocytes, the donor eGFP⁺ cells also expressed the hepatic markers albumin and CK-18 (FIG. 3C, middle and right) marked by white arrows. mo: month, magnifications: albumin and CK-18 (20×), vWF (40×), FISH (100×).

A flow cytometric analysis revealed that the majority of eGFP⁺ cells expressed albumin, further ensuring that they were directly differentiated from BMCs (FIG. 4G). As further support of direct differentiation, we show two binucleated cells that are donor-derived hepatocytes (FIGS. 4H and 4I). Donor cell shows two discrete nuclei and the cell expressed CK-18. It may be noted that both the nuclei in the cell are distinct, like in recipient hepatocytes. Together, the results of FISH and immunocytochemistry support the notion that BMCs directly differentiated into hepatocytes.

FIG. 4G flow cytometric analysis of control (untransplanted) and HAT mice liver cells. Representative dot-plots show no eGFP+ cells in the liver of control mouse (left), whereas in HAT mouse 8.8% cells were eGFP+ and 97% of them expressed albumin (right). FIG. 4H shows a comparison between the binucleated cell of recipient and donor derived hepatocyte. Donor cell shows two discrete nuclei and the cell expressed albumin. FIG. 4I is a comparison between the binucleated cell of recipient and donor derived hepatocyte. Donor cell shows two discrete nuclei and the cell expressed CK-18. A similar binucleated cell is shown in the recipient hepatocytes.

Quantitative analyses of donor-derived hepatic and endothelial cells were carried out by flow cytometry. Quantitative analysis of GFP+Albumin+ and GFP+vWF+ cells at different times of transplantation are shown in FIGS. 4A, 4B, and 4C. A single-cell suspension of liver was analyzed by flow cytometry. FIG. 4A shows representative dot-plots for GFP⁺, Albumin⁺ and GFP⁺, vWF⁺ cells at different times. In the top panel, R1, R2, and R3 are the regions for eGFP⁺, Albumin⁺, and GFP+Albumin+ cells, respectively. In the bottom panel, R1, R2, and R3 are the regions for eGFP+, vWF+, and GFP+vWF+ cells, respectively.

FIGS. 4B shows the percentage of eGFP+ cells that express albumin at different times. The results show that more than 85% of donor-derived cells (eGFP+) expressed albumin in all three time points examined (n=4, for each time point).

FIG. 4C shows the percentage of eGFP+ cells that express vWF at different times. The results show that 4-8% of donor-derived cells expressed vWF (n=4, for each time point).

To study normal organ function the serum ALT level for the liver, and the plasma creatinine and BUN levels for the kidney of HAT mice were determined, and were compared with levels from healthy mice. The biochemical parameters tested in six mice between 1 and 6 mo after transplantation showed normal organ functions. (FIG. 4D). No macroscopic changes were noticed in the livers and kidneys of the control and HAT mice during the study as compared to WT mice (FIG. 4J). These results likely represent the normal physiological functions of the liver and kidney in posttransplanted HA mice.

### Bmc Transplantation Resulted in FVIII Light Chain Protein Expression in HA Mice

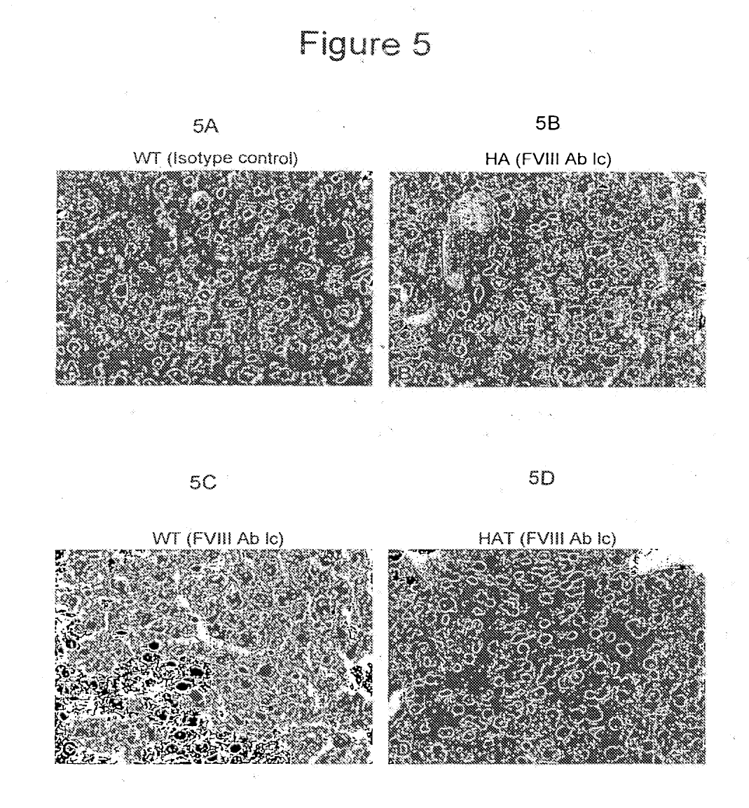

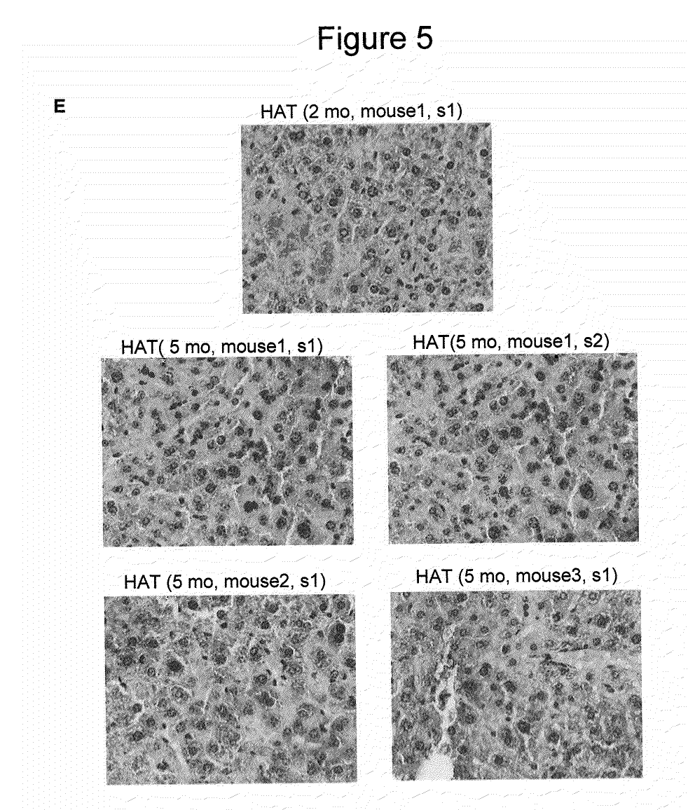

Representative photomicrographs show the expression of the FVIII light chain protein in WT and HAT mice. Paraffin-embedded serial liver sections of WT, HA, and HAT (5 mo of transplantation) mice were stained and examined under a microscope (magnification 40×). The light chain antibody staining was specific as no reaction was observed with isotype control antibody. The expression of FVIII in the liver tissue was examined by IHC. The mutant mouse used in this investigation has a disrupted exon 16 within the A3 domain of the FVIII gene, and the intact light chain protein is thus not produced, whereas the heavy chain protein is expressed (data not shown). To determine whether HAT mice express the FVIII light chain protein, serial liver sections of control and transplanted mice were stained with either isotype control or specific antibodies. FIG. 5A shows WT mice. The wild type (WT) mice showed expression of the FVIII-light chain throughout the section (FIG. 5C). The same protein was not detected in the liver section of HA mice (FIG. 5B) (Light chain staining in HA mice).

The FVIII-light chain protein expression was observed in HAT mice. This would not be possible unless BMCs differentiated into competent liver cells. The expression of the FVIII light chain protein was observed to the centrilobular and other parts of the liver section as well (FIGS. 5D and 5E). No background staining was observed with the isotype control antibody, indicating that the above staining was specific.

Paraffin-embedded serial liver sections of HA and HAT (after 2 and 5 mo of transplantation) mice were with FVIII-light chain antibody followed by goat anti-rabbit IgG-HRP, and finally the sections were reacted with DAB. Paraffin-embedded serial liver sections of HA and HAT (after 2 and 5 mo of transplantation). No background staining was observed with the isotype control antibody, indicating that the above staining was specific. Representative photomicrographs show the expression of the FVIII light chain protein in HAT mice.

### Bleeding Disorder is Corrected in HA Mice

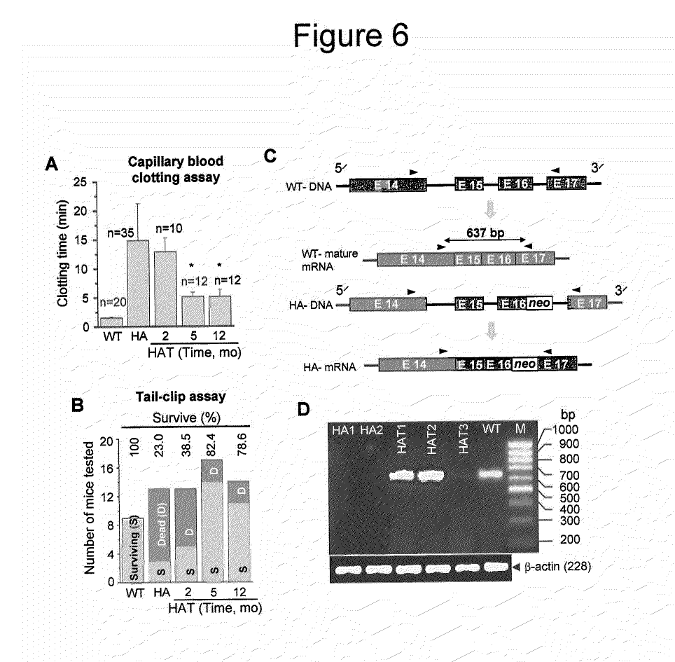

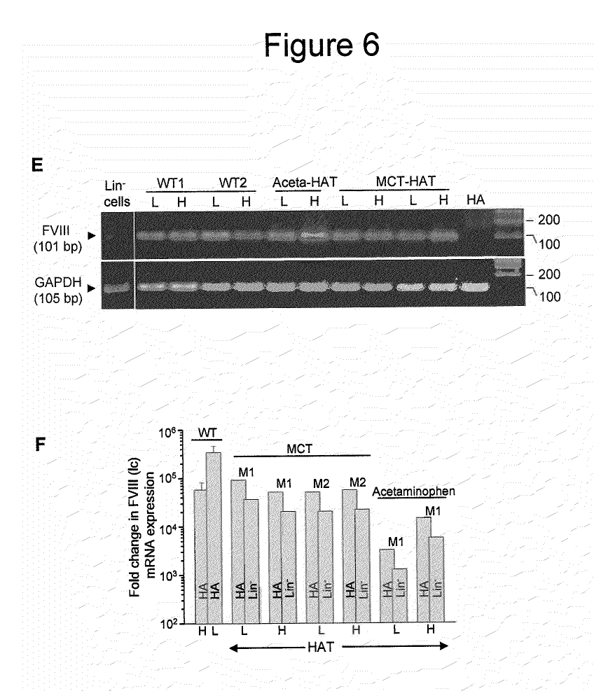

The phenotypic correction in the HA mice was determined by blood clotting and tail-clip challenge experiments. Blood was withdrawn from the eye in a capillary tube. The clotting time was determined by visual observation of the clot formation. The results showed a significant drop in the blood clotting time in HAT mice after 5 mo after transplantation as compared to the HA mice. The number of mice tested (n) in each condition is mentioned in the figure (*p<0.01). The whole blood clotting time in WT mice was 1.9±0.3 min (n=20), which was increased to 14.9±6.5 min (n=35) in HA mice. The whole blood clotting time was significantly (p<0.01, n=24) reduced 5 mo after transplantation (FIG. 6A).

Tail clip challenge. The numbers of surviving and dead mice are shown after tail clip in FIG. 6B. The survival rate in HAT mice increased from 23 to 80% in the tail-clip challenge experiment. Twenty-five of 31 HAT mice (5 and 12 mo after transplantation) that received Lin⁻ BMCs achieved hemostasis at 4 h or less, and survived to the end of the study. The mice that did not stop bleeding died within 10 to 20 h of the tail-clip. It was found that the majority of HA mice did not survive more than 20 h after the tail-clip. In HAT mice, 80% were protected from death due to blood loss. The numbers of mice tested in each group are as follows: WT (9 mice), HA (13 mice), HAT-2 mo (13 mice), HAT-5 mo (17 mice), and HAT-12 mo (14 mice).

The results of these functional assays suggest that BMCs not only produced FVIII protein in liver of HA mice, but also that the protein was secreted into the circulation.

The rationale for designing primers to amplify a sequence of the FVIII A3 domain that it is difficult to reverse transcribe across the neo sequence due to the presence of high G+C content in HA mice (FIG. 6C).

To confirm the phenotypic rescue, we analyzed the gene expression of the FVIII light chain by RT-PCR, as the intact light chain is missing in HA mice. We designed a primer pair to amplify a specific segment of the light chain A3 domain where the gene disruption was introduced in the mutant mouse (FIG. 6C). PCR amplification of the wild type cDNA yielded a 637-bp product. However, due to the presence of the neo cassette sequence (high G+C content) it is difficult to reverse transcribe and amplify the corresponding product in HA mice. The results showed that the same gene fragment was amplified in WT mice, but not in mutant (HA1 and HA2) mice (FIG. 6D). Intriguingly, the target gene fragment was amplified in all three HAT mice. HAT1-3 mice show the synthesis of a 637 by amplicon, the same as in wild type mice. The same gene product was absent in both the knock-out mice (HA1 and HA2).

To confirm that FVIII gene is expressed by hepatocytes and endothelial cells and to compare the relative expression, we conducted real-time RT-PCR analysis of light chain mRNA in both cell fractions. Hepatocytes and LSECs were separated from the perfused liver cells of WT and HAT mice using standard protocols. Hepatocytes and endothelial cells were isolated from WT and HAT mice, and the mRNA was subjected to RT-PCR using primers designed for real-time RT-PCR. As negative controls, HA mice whole liver and Lin⁻ BMCs mRNA were used. The results suggest that both hepatocyte and endothelial cell fractions expressed FVIII (lc) gene because a 101-bp fragment was amplified (FIG. 6E). Further, the expression of same gene was discernible in the Lin⁻ BMCs. In real-time RT-PCR experiments, relative signals of the test samples were determined with respect to the signal of the HA (whole liver) and Lin⁻ BMCs. Hepatocytes and LSECs isolated from WT mice showed extremely high relative expression of the target amplicon as gene expression by HA mice was negligible (FIG. 6F). Among these two cell types, LSECs expressed about 5-fold more FVIII (lc) mRNA (FIG. 6F). In MCT-HAT mice, the expression of the gene product in hepatocytes and LSECs was comparable, despite variability from mouse to mouse. Similarly, in the case of acetaminophen-HAT mice, FVIII (lc) mRNA was expressed by both cell types, although the fold expression was lower than for MCT-HAT mice (FIG. 6F). In case of Lin⁻ BMCs serving as control, the relative expressions of the gene were much lower as compared to the HA (FIG. 6F). Taken together, this RT-PCR and real-time RT-PCR data suggest that FVIII (lc) mRNA is expressed by both hepatocytes and LSECs of WT mice. Furthermore, in both MCT- and acetaminophen-induced liver injury models, BMCs-derived hepatocytes and LSECs expressed the FVIII (lc) gene.

TEM Confirms that Both Hepatocytes and LSECs Express FVIII (lc) Protein

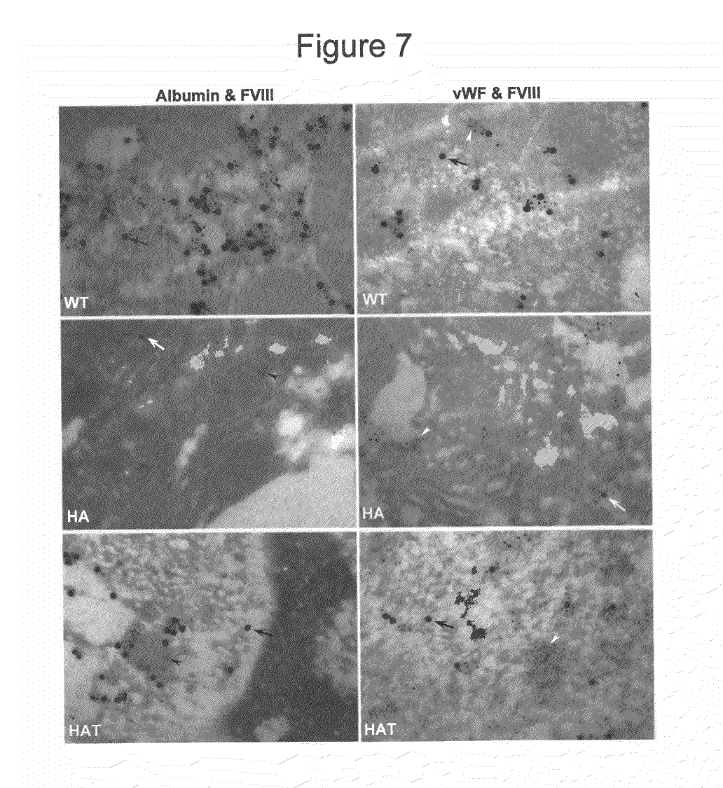

Post-embedded immunogold-labeled transmission electron micrographs also confirmed the above finding that FVIII (lc) protein is expressed by hepatocytes as well as by endothelial cells. WT, HA and HAT mouse liver sections were either stained with anti-albumin and anti-FVIII (lc) or anti-vWF and anti-FVIII (lc) antibodies. It may be noted that HA mouse liver sections showed albumin and vWF staining, whereas FVIII (lc) protein was not expressed (FIG. 7, middle panel). We observed 1 to 2 colloidal gold particles, targeted to FVIII (lc) protein, were nonspecifically attached to the HA mouse liver sections. In WT and HAT mice, co-expression of albumin and FVIII (lc) proteins was observed in the cytoplasm of the target cells (FIG. 7, left upper and lower panel). These results indicated that FVIII protein is expressed by hepatocytes. Similarly, vWF-expressing cells also synthesized FVIII protein in WT and HAT mice, confirming that endothelial cells are also a source of FVIII (FIG. 7, right upper and lower panel).

### Plasma FVIII Activity and Inhibitor Level in HAT Mice

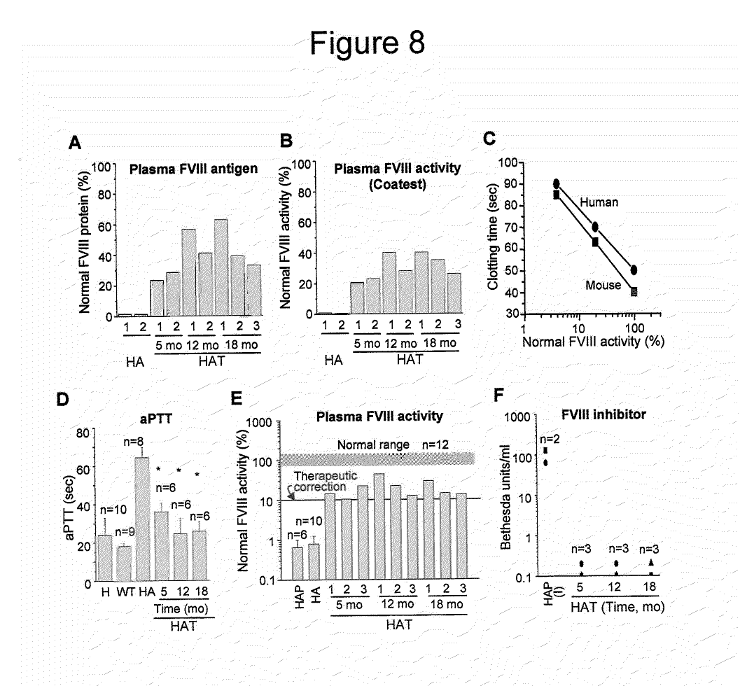

The results of the whole blood clotting and tail-clip challenge experiments in HAT mice suggest that the FVIII protein was present in the blood. We performed an ELISA using a specific antibody to determine the relative FVIII antigen level in the plasma, which varied from mouse to mouse (FIG. 8A). The average antigen levels during the study were 41.7±5.4% (n=7) of normal FVIII protein. Again, to evaluate the therapeutic effect of BMCs, the same plasma samples from HAT mice were analyzed by COATEST assay (FIG. 8B). FIG. 8B shows the relative FVIII activity in HAT mice.

The standard curve of normal pooled plasma FVIII activity versus clotting time in second was determined. The average FVIII activities in plasma collected from 5 to 18 mo after transplantation were 30.6±3.0% (n=7) of normal plasma. The phenotypic rescue in HAT mice was further examined by an aPTT assay. Since the aPTT assay reagent is customarily used for human samples, we initially tested both mouse and human pooled plasma and the correlation between these samples was determined. It appears that the aPTT reagent responded similarly with mouse and human plasma FVIII (FIG. 8C). The aPTT values of pooled human and WT, HA, HAT mice are shown. The aPTT values of WT mice were 15.5 to 19.3 sec, which was increased to 62.5 to 75.3 sec in HA mice. The aPTT values were significantly lower (25.3 to 41.6 sec) in HAT mice (* p<0.01).

There was a 3-fold increase in the aPTT time in HA mice compared to the WT mice. The aPTT value was significantly (p<0.01) decreased in HAT mice, supporting our tail-clip challenge results (FIGS. 6B and 8D).

In WT mice, the plasma FVIII activity was 96±15% (n=12) as compared to the normal pooled mouse plasma. The FVIII activity in HA mice was 0.7±0.25%, which was increased to 15.7±3.2%, 26.4±9.4%, and 19.1±5.1% in HAT mice after 5, 12, and 18 mo of transplantation, respectively (FIG. 8E). The relative percentage of FVIII activities in plasma samples from HAT mice and patients were determined. The normal range of mouse FVIII activity is indicated by the gray shaded region. A Tukey-Kramer multiple comparison analysis concluded that the plasma FVIII activity in HAT mice was significantly (HA to HAT₅ₘₒ: p<0.05; HA to HAT₁₂ₘₒ: p<0.001; HA to HAT₁₈ₘₒ: p<0.01) higher in all three test groups as compared to the HA mice. These results indicated that the average FVIII activity from 5 to 18 mo of study was stable at 20.4±3.6% (n=9) (FIG. 8E). Thus, the FVIII activity by COATEST assay was 1.5-fold higher than observed by aPTT assay.

HA mice frequently develop inhibitors after FVIII protein or gene therapy. Therefore, the transplanted mice were tested to determine if they developed inhibitors to FVIII. Nine of nine HAT mice examined between 5 to 18 mo of study confirmed that the BM cell therapy did not cause the formation of FVIII inhibitors (FIG. 8F).

FIG. 8F shows FVIII inhibitors in HAT mice and patients plasma. Mice and patients plasma samples, described in regard to FIG. 8E, were tested for anti-FVIII activity by the Bethesda assay. The results show the absence of inhibitor in all the plasma samples of HAT mice. mo: month, H: normal human, WT: wild type mouse, HA: hemophilic A mouse, HAP: hemophilic A patient, HAP (I): hemophilic A patient-inhibitor.

Injury with Monocrotalin

### Materials and Methods

Animals: Six to eight weeks old male HA mice [B6; 129S4-F8^{tm/Kaz}/J] and GFP expressing [C57BL/6-Tg(UBC-GFP)₃₀Scha/J] female mice were used in this investigation. Mice were obtained from The Jackson Laboratories (Bar Harbor, Me.) and maintained in the institute's experimental animal facility. Mice were kept in an isolator, fed with autoclaved acidified water and irradiated food ad libitum. All experiments were conducted as per procedures approved by the Institutional Animal Ethics Committee.

Conditioning of liver: Liver was conditioned by intraperitoneal injection of monocrotalin (200 mg/kg b.w.) in male HA mice. Lin BMCs (0.25×10⁶ cells/mice) from GFP transgenic female mice was injected to the above mice through intravenous route 24 to 48 h after the monocrotalin treatment. Mice was held for different times before analysis.

Blood clot and plasma analysis: Capillary blood clotting, tail-clip, plasma FVIII activity (aPTT and coatest based) assays were essentially performed following the same protocols set out above.

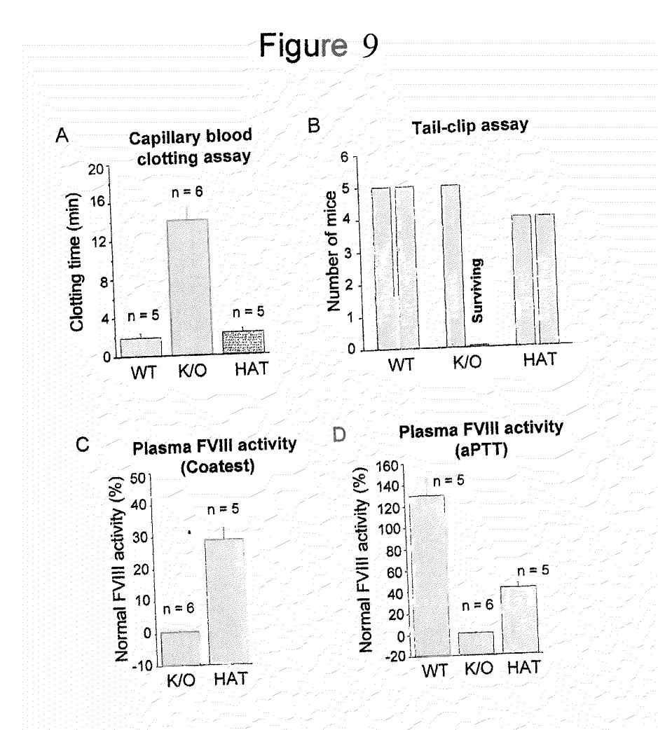

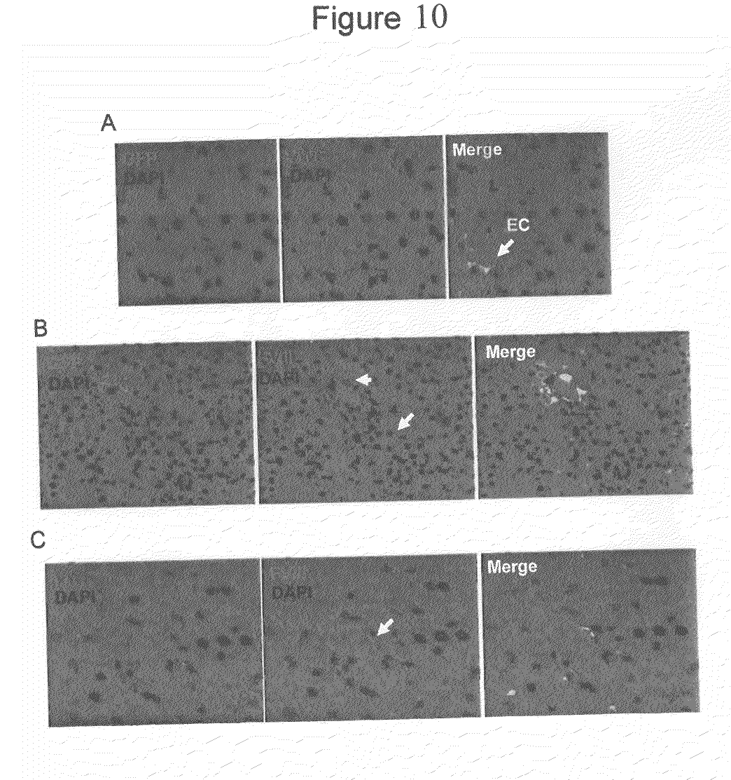

FIG. 9A shows the results from whole blood clotting assay. Blood was withdrawn from eye in a capillary tube. The clotting time was determined by visual observation of clot formation. Results showed significant drop in blood clotting time in HAT mice after 5 months of transplantation as compared to the HA mice. Number of mice tested (n) in each condition are mentioned in the figure (*p<0.01).

FIG. 9B shows the results from the tail clip challenge. The number of surviving mice are shown after tail clip. In HAT mice, 100% were protected from death due to blood loss.

FIG. 9C shows relative FVIII activity in HAT mice. The FVIII activity was determined by the COATEST assay as described above.

FIG. 9D shows relative FVIII activity. The relative percentage of FVIII activities in plasma samples from HAT mice was determined on aPTT-based assay. WT: wild type mouse, HA: hemophilic A mice, HAT: hemophilic A mice transplanted.

Immuniohistochemical analysis: Liver tissues were fixed in 4% paraformaldehyde, cryoprotected in 30% sucrose solution for 24 h at 4° C., and frozen in tissue freezing medium. Five-micron serial sections were treated with 0.15% Triton X-100 for 30 min at room temperature. The sections were then stained with rabbit anti-GFP at 1:200, anti-vWF (1:100), and anti-FVIII (1:200) antibodies for 1 h at room temperature. The secondary antibodies were conjugated with AlexaFluor 488/594 (Molecular Probes Inc., OR, USA). Cells were imaged under an Olympus fluorescence microscope using LCPlanFl 40× objectives and under an Olympus Fluoview1000 confocal laser scanning microscope using PlanApomat 100× oil immersion objective, and a DP70 digital camera. Olysia Bioreport and FlowView 5.0 were the software used for image acquisition. Images were composed and edited in Image ProPlus and Photoshop 6.0 (Adobe).

Five months after transplantation, mice were sacrificed and liver cryosections were analyzed by IHC. Five-micron serial sections were stained with anti-GFP, anti-vWF, and anti-FVIII antibodies. FIG. 9A shows Confocal images. The merged picture shows that donor-derived (eGFP)⁺ cells express vWF. FIG. 9B is also a merged picture that shows that donor-derived (eGFP)⁺ cells express FVIII light chain protein. FIG. 9C is a merged picture that shows that vWF expressing (endothelial) cells express FVIII light chain protein.

Overall, these findings were in agreement with the maintenance of the secretory functions of BM-derived liver cells and the ability of these cells to supply active FVIII in HA mice.

TABLE 1A
The Primer Sequences, Amplicon Sizes And Annealing
Temperatures Of Various PCR Products

| | Sense | Anti-sense | Amplicon | Ta |
|---|---|---|---|---|
| Gene | primer | primer | size (bp) | (° C.) |
| Transforming | CAGCATGTGTC | GTGTTGTCCCA | 350 | 62 |
| growth factor | TGCCACTCT | AGATGCCTT | | |
| α(TGFα) | (SEQ ID NO: 1) | (SEQ ID NO: 2) | | |
| Fetal liver | CAGACCAACAT | GGTTCCCAACT | 518, 580 | 60.8 |
| tyrosine kinase- | CTCCCACCT | CTAGCACCA | | |
| 3 ligand (Flt3-L) | (SEQ ID NO: 3) | (SEQ ID NO: 4) | | |
| Interleukin-6 | GTTCTCTGGGA | TGGTCTTGGTC | 397 | 57.9 |
| (IL-6) | AATCGTGGA | CTTAGCCAC | | |
| | (SEQ ID NO: 5) | (SEQ ID NO: 6) | | |
| Stem cell factor | TCCGAAGAGG | CACGGGTAGCA | 501, 585 | 63.0 |
| (SCF) | CCAGAAACTA | AGAACAGGT | | |
| | (SEQ ID NO: 7) | (SEQ ID NO: 8) | | |
| Vascular | AGCACAGCAG | CCTCTTCCTTC | 221, 353, | 62 |
| endothelial growth | ATGTGAATGC | ATGTCAGGC | 425 | |
| factor a (VEGFa) | (SEQ ID NO: 9) | (SEQ ID NO: 10) | | |
| Stromal derived | TTTCACTCTCG | AGATGCTTGAC | 215 | 61.5 |
| factor-1α (SDF1α) | GTCCACCTC | GTTGGCTCT | | |
| | (SEQ ID NO: 11) | (SEQ ID NO: 12) | | |
| Hepatocyte growth | CTACACTCTTG | GCCACGATAAC | 370 | 59.4 |
| factor (HGF) | ACCCTGACAC | AATCTTGTCC | | |
| | (SEQ ID NO: 13) | (SEQ ID NO: 14) | | |
| FVIII-A3 domain | TCAAATCCCAC | GGGTCCAATTA | 637 | 57 |
| | CAGTGTTGA | ATCCCGAGT | | |
| | (SEQ ID NO: 15) | (SEQ ID NO: 16) | | |
| B-Actin | AGCCATGTACG | CTCTCAGCTGT | 228 | 58.8 |
| | TAGCCATCC | GGTGGTGAA | | |
| | (SEQ ID NO: 17) | (SEQ ID NO: 18) | | |

TABLE 1B
Primers sequences, amplicon sizes and annealing
temperatures, and cycle number of PCR reactions

| | | | Annealing | |
|---|---|---|---|---|
| | Primer | Amplicon | temperature | Cycle |
| Gene | sequences | size (bp) | (° C.) | number |
| RT-PCR: FVIII | TCAAATCCCACCAGT | 637 | 57 | 30 |
| (A3 domain) | GTTGA (forward) | | | |
| | (SEQ ID NO: 19) | | | |
| | GGGTCCAATTAATCCC | | | |
| | GAGT (reversed) | | | |
| | (SEQ ID NO: 20) | | | |
| RT-PCR & Real-time | GCCTGGGCTTATTTCT | | 60 | 30 (RT- |
| PCR: FVIII | CTGATG (forward) | 101 | | PCR) |
| (A3 domain) | (SEQ ID NO: 21) | | | |
| | TGAGCAGGATTCAGT | | | |
| | GTGTTCG (reversed) | | | |
| | (SEQ ID NO: 22) | | | |
| RT-PCR & Real- | CCTCGACCACAACTG | 102 | 60 | 30 (RT- |
| time PCR: GAPDH | CTTAG (forward) | | | PCR) |
| | (SEQ ID NO: 23) | | | |
| | GGGTGGCAGTGATGG | | | |
| | CAT (reversed) | | | |
| | (SEQ ID NO: 24) | | | |

TABLE 2
Metaphase cytogenetics of HAT liver cells

| | |
|---|---|
| Total nuclei counted | 4454 |
| Donor nuclei (only X bearing discrete nuclei; no Y) | 421 |
| Normal donor cells (%) | 9.45 |
| Recipient nuclei (Y bearing discrete nuclei; with X) | 4025 |
| Normal recipient cells (%) | 90.35 |
| Recipient (XY presence) + Donor (Y absent, presence | 8 |
| of X signals only) | |
| Fused/overlapped/abnormal cells (%) | 0.2 |

### REFERENCES

1. Lozier I N, Kessler, C M. In: Hematology: Basic Principles and Practice. Hoffman, R., Benz, E J, Shattil, S J, Furie, B, Cohen H J, Silberstein, L E, McGlave, P, eds. New York, Churchill: Livingstone; 2000: 1883-1904.
2. Greengard J S, Jolly D J. Animal testing of ret rovira I-mediated gene therapy for factor VIII deficiency. Thromb Haemost. 1999; 82: 555-561.
3. Park F, Ohashi K, Kay M A. Therapeutic levels of human factor VIII and IX using HIV-1 based lentiviral vectors in mouse liver. Blood. 2000; 96: 1173-1176.
4. Chuah M K L, Schiedner G, Thorrez L, et al. Therapeutic factor VIII levels and negligible toxicity in mouse and dog models of hemophilia A following gene therapy with high-capacity adenoviral vectors. Blood. 2003; 101:1734-1743.
5. Matsui H, Shibata M, Brown B, et al. Ex Vivo gene therapy for hemophilia A that enhances safe delivery and sustained In vivo factor VIII expression from lentivirally engineered endothelial progenitors. Stem Cells. 2007; 25: 2660-2669.
6. Manno C S, Chew A J, Hutchison S, et al. AA V-mediated factor IX gene transfer to skeletal muscle in patients with severe hemophilia B. Blood. 2003; 101:2963-2972.
7. Manno C S, Pierce G F, Arruda Y R, et al. Successful transduction of liver in hemophilia by AA V-Factor IX and limitations imposed by the host immune response. Nat Med. 2006; 12: 342-347.
8. Wion K L, Kelly D, Summerfield J A, et al. Distribution of factor VIII mRNA and antigen in human liver and other tissues. Nature. 1985; 317: 726-729.
9. Shaw E, Giddings J C, Peake I R, Bloom A L. Synthesis of procoagulant factor VIII, factor VIII-related antigen, and other coagulation factors by the isolated perfused rat liver. Br J Haemato L 1979; 41: 585-596.
10. Owen C A, Bowie E J W, Fass, D N. Generation of factor VIII coagulant activity by isolated, perfused neonatal pig livers and adult rat livers. Br J Haemato L 1979; 43: 307-315.
11. Marchloro T L, Hougie C, Ragde H, Epstein R B, Thomas E D. Hemophilia: role of organ homografts. Science. 1969; 163:188-190.
12. Lewis J H, Bontempo F A, Spero J A, Ragni M V, Starzl T E. Liver transplantation in a hemophiliac. N Engl J Med. 1985; 312:1189-1190.
13. Bontempo F A, Lewis J H, Gorenc T J, et al. Liver transplantation in hemophilia A. Blood. 1987; 69:1721-1724.
14. Figueiredo M S, Brownlee G G. cis-Acting elements and transcription factors involved in the promotor activity of the human factor VIII gene. J Biol Chem. 1995; 270:11828-11838.
15. Zelechowska M G, van Mourik J A, Brodniewicz-Proba T. Ultrastructure localization of factor VIII procoagulant antigen in liver hepatocytes. Nature. 1985; 317: 729-730.
16. Ohashi K, Waugh I M, Dake M D, et al. Liver tissue engineering at extrahepatic sites in mice as a potential new therapy for genetic liver diseases. Hepatology. 2005; 41: 132-140.
17. Do H, Healey J F, Waller E K, Lollar P. Expression of factor VIII by murine liver sinusoidal endothelial cells. J Biol Chem. 1999; 274:19587-19592.
18. Follenzi A, Benten D, Novikoff P, Faulkner L, Raut S, Gupta S. Transplanted endothelial cells repopulate the liver endothelium and correct the phenotype of hemophilia A mice. J Clin Invest. 2008; 118: 935-945.
19. Lagasse E, Connors H, AI-Dhalimy M, et al. Purified hematopoietic stem cells can differentiate into hepatocytes in vivo. Nature Med. 2000; 6:1229-1234.
20. Wang X, Ge S, McNamara G, Hao Q L, Crooks G M, Nolta J A. Albumin-expressing hepatocyte-like cells develop in the livers of immune-deficient mice that received transplants of highly purified human hematopoietic stern cells. Blood. 2003; 10 1: 4201-4208.
21. Jang Y, Collector M I, Baylin S B, Diehl A M, Sharkis S J. Hematopoietic stem cells convert into liver cells within days without fusion. Nature Cell Biol. 2004; 6:532-539.
22. Khurana S, Mukhopadhyay A. Characterization of the potential subpopulation of bone marrow cells involved in the repair of injured liver tissue. Stem Cells. 2007; 25: 1439-1447.
23. Khurana S, Mukhopadhyay A. In vitro transdifferentiation of adult hematopoietic stem cells: an alternate source of hepatocytes. J Hepatol. 2008; 49: 998-1007.
24. Wang Y J, Li M D, Wang Y M, Nie Q H, Chen G Z. Experimental study of bioartificial liver with cultured human liver cells. World J Gastroenteroi. 1999; 5:135-137.
25. Connelly S, Andrews J, Gallo A. Sustained phenotypic correction of murine hemophilia A by in vivo gene therapy. Blood. 1998; 91: 3273-3281.
26. Voigt G L. In: Hematology Techniques and Concepts for Veterinary Technicians. Blackwell Publishing; 2000: 109.
27. Zacharski L R, Rosenstein R. Standardization of the one-stage assay for factor VIII (antihemophilic factor). Am J Clin Pathoi. 1978; 70: 280-286.
28. Forarty P F, Rick M E. Disorder of hemostasis I: Coagulation. In: Bethesda Handbook of Clinical Hematology. Rodgers G P, Young N S, eds. Lippincott Williams and Wilkins; 2005: 272.
29. Bi L, Sarkar R, Naas T, et al. Further characterization of Factor VIII-deficient mice created by gene targeting: RNA and protein studies. Blood. 1996; 88: 3446-3450.
30. Sarkar R, Gao G P, Chirmule N, Tazelaar J, Kazazian H H. Partial correction of murine hemophilia A with non-antigenic murine factor VIII. Hum Gene Ther. 2000; 11: 881-894.
31. O'Mahony B, Black C. Expanding hemophilia care in developing countries. Seminars in Thromb Hemost. 2005; 31: 561-568.
32. Gan, Uin S, Lian. K O, Roy C. Genetic engineering for haemophilia A. Exp Opin Biol Ther. 2006; 6: 1023-1030.
33. High K A. The leak stops here: platelets as delivery vehicles for coagulation factors. J Clin Invest. 2006; 116:1840-1842.
34. Mannucci, P M. Hemophilia: treatment options in the twenty-first century. J Thromb Haemost. 2003; 1:1349-1355.
35. Tatsumi K, Ohashi K, Shima M, Nakajima Y, Okano T, Yoshioka A. Therapeutic effects of hepatocyte transplantation on hemophilia B. Transplantation. 2008 15; 86:167-170.
36. Petersen B E, Bowen W C, Patrene K D, et al. Bone marrow as a potential source of hepatic oval cells. Science. 1999; 284: 1168-1170.
37. Theise N D, Badve S, Saxena R. Derivation of hepatocytes from bone marrow cells in mice after radiation-induced myoablation. Hepatology. 2000; 31:235-240.
38. Schwartz R E, Reyes M, Koodie L, et al. Multipotent adult progenitor cells from bone marrow differentiate into functional hepatocyte-like cells. J Clin Invest. 2002; 109:1291-1302.
39. Makin A J, Williams R. Acetominaphen-induced acute liver failure. In: Acute liver failure. Lee W M, Williams R, eds. Cambridge, UK: Cambridge University Press; 1997: 33.
40. Schmidt C, Bladt F, Goedcke S, et al. Scatter factorihepatocyte growth factor is essential for liver development. Nature. 1995; 373: 699-702.
41. Webber E M, Wu J C, Wang L, Merlino G, Fausto N. Over expression of transforming growth factor-alpha causes liver enlargement and increased hepatocyte proliferation in transgenic mice. Am J Pathol. 1994; 145: 398-408.
42. Hollestelle M J, Thinnes T, Crain K, et al. Tissue distribution of FVIII gene expression in vivo: a closer look. Thromb Haemost. 2001; 86: 855-861.
43. Doering C B, Josephson C D, Craddock R N, Lollar P. Factor VIII expression in azoxymethane-induced murine fulminant hepatic failure. Blood. 2002; 100: 143-147.
44. Jacquemin M, Neyrinck A, Hennanns M I, et al. FVIII production by human lung microvascular endothelial cells. Blood. 2006; 108: 515-517.
45. Lau A H, Creus Ade, Lu L, et al. Liver tolerance mediated by antigen presenting cells: factor fictions? Gut. 2003; 52, 1075-1078.
46. Crispe I N. Hepatic T cells and liver tolerance. Nat. Reviews 2003; 3, 51-62.
47. Lucienne M I, Bagirath G, Kuang-Yueh C, et al. Hematopoietic stem-cell gene therapy of hemophilia A incorporating a porcine factor VIII trans gene and nonmyeloablative conditioning regimens. Blood. 2007; 110:2855-2863.
48. Hoeben R C, Fallaux F J, van Tilburg N H, et al. Toward gene therapy for hemophilia A: longterm persistence of factor VIII-secreting fibroblasts after transplantation into immunodeficient mice. Hum. Gene Ther. 1993; 4: 179-186.
49. Wang X, Willenbring H, Akkari Y, et al. Cell fusion is the principal source of bone-narrow derived hepatocytes. Nature. 2003; 422: 897-901.
50. Vassilopoulos G, Wang P, Russell D W. Transplanted bone marrow regenerates liver by cell fusion. Nature. 2003; 422: 901-904.
51. Vignery A. Osteoclasts and giant cells: macrophage-macrophage fusion mechanism. Int. J. Exp. Pathol. 2000; 81: 291-304.
52. Mintz B, Baker W W. Normal mammalian muscle differentiation and gene control of isocitrate dehydrogenase synthesis. Proc. Natl. Acad. Sci. USA 1967; 58: 592-598.
53. Yang T C, Georgy K A, Craise L M, Durante M. Initiation of Oncogenic Transformation in Human Mammary Epithelial Cells by Charged Particles. Rad. Onco. Invest. 1997; 5: 134-138.
54. Hasegawa R, Furukawa F, Toyoda K, et al. Study for tumor-initiating effect of acetaminophen in two-stage liver carcinogenesis of male F344 rats. Carcinogenesis 1988; 9: 755-759.

1. A method to control bleeding in a subject having hemophilia comprising administering bone-marrow cells converted into hepatocytes and endothelial cells in an amount sufficient to produce Factor VIII in said subject.

2. The method according to claim 1 wherein Factor VIII is expressed at levels having a therapeutic effect on said subject and wherein said therapeutic effect is an increase in coagulation of blood.

3. The method according to claim 1, wherein the hemophilia is hemophilia A.

4. The method according to claim 1, wherein the hemophilia is hemophilia B.

5. The method according to claim 1, wherein 20 to 25×10⁶ hepatocytes are administered to the subject.

6. The method according to claim 1, wherein 10 to 25×10⁷ bone marrow cells are administered to the subject.

7. The method according to claim 5 wherein the cells are administered in a single dose.

8. The method according to claim 5 wherein the cells are administered in multiple doses.

9. The method according to claim 1 wherein the subject is a human.

10. The method according to claim 1 wherein the bone-marrow cells are selected from the group consisting of mononuclear cells, Lin⁻ cells, mesenchymal stem cells and hematopoietic stem cells.

11. A method for treating a clotting disorder comprising administering hepatocytes derived from bone-marrow cells in an amount sufficient to produce Factor VIII to a patient in need thereof wherein Factor VIII is expressed at levels having a therapeutic effect on said patient.

12. A method for treating hemophilia A in an individual in need thereof comprising administering to the individual an amount of a composition comprising hepatocytes from bone-marrow cells sufficient to produce FVIII wherein Factor VIII is expressed at levels having a therapeutic effect on said patient.
